(19) United States
(12) Patent Application Publication
(10) Pub. No.: US 2021/0079079 A1
(43) Pub. Date: Mar. 18, 2021

(54) SYSTEMS AND METHODS FOR LOCAL MODULATION OF WNT SIGNALING

(71) Applicant: Ortheus, Inc., Newbury Park, CA (US)

(72) Inventors: Paul Kostenuik, Newbury Park, CA (US); Faisal Mirza, Saratoga, CA (US); Michael Collins, San Marcos, CA (US)

(73) Assignee: Ortheus, Inc., Newbury Park, CA (US)

(21) Appl. No.: 17/049,567

(22) PCT Filed: May 1, 2019
(86) PCT No.: PCT/US2019/030233

§ 371 Date: Oct. 21, 2020

Related U.S. Application Data
(60) Provisional application No. 62/665,981, filed on May 2, 2018.

(51) Int. Cl. C07K 16/18; A61K 35/16; A61K 35/19; A61K 35/28; A61K 35/32; A61K 38/17

(52) U.S. Cl. CPC C07K 16/18; A61K 35/16; A61K 35/19; A61K 9/0019; A61K 35/32; A61K 38/1709; A61K 35/28

(57) ABSTRACT

A system is provided including a promoter of Wnt signaling and an autologous body material (ABM) or its functional equivalent. The promoter of Wnt signaling can be an agent that inhibits the activity or bioavailability of DKK1 protein, e.g., an anti-DKK1 antibody (DKAB). The promoter of Wnt signaling can also be an agent that initiates, promotes, or potentiates Wnt signaling by means other than inhibition of DKK1. The ABM in some embodiments are prepared to remove DKK1 or an antagonist of Wnt signaling. A process of locally administering the system is also provided to enhance the intended or ancillary effects of the ABM. The system is useful in promoting the growth of new bone or the augmentation, reconstitution, regeneration, fusion, fixation, repair, or healing of damaged, injured or otherwise deficient bone in therapeutically or esthetically desirable locations, or for promoting hair growth or wound healing.

### CROSS-REFERENCE TO RELATED APPLICATIONS

This application includes a claim of priority under 35 U.S.C. § 119(e) to U.S. provisional patent application No. 62/665,981, filed May 2, 2018, the entirety of which is hereby incorporated by reference.

### FIELD OF INVENTION

This invention relates to systems and methods for enhancing Wnt signaling in therapeutic autologous body materials (ABMs) and their functional equivalents, and at sites upon or within the body where those materials are administered, to promote the growth, healing, repair, regeneration, and augmentation of tissues.

### BACKGROUND

A variety of autologous materials have been developed for therapeutic applications, including those that promote skeletal and soft-tissue healing, repair, regeneration, and augmentation. Autologous materials have been extensively studied and used therapeutically in orthopedics, plastic surgery, craniomaxillofacial surgery, dentistry, wound healing, hair restoration, and other settings. Autologous materials include whole blood; platelets; platelet-rich therapies (PRTs) including platelet gel, platelet-rich plasma (PRP), platelet-poor plasma (PPP) platelet-rich fibrin (PRF), leukocyte-rich PRP and PRF (L-PRP and L-PRF, respectively); fibrin gel; serum and hyperacute serum; various pluripotent stem cells including mesenchymal stem cells (MSCs) and other cellular fractions from various tissue sources including autografts; bone marrow, bone marrow aspirate (BMA), bone marrow aspirate concentrate (BMAC) reamer-irrigator aspirate (MA); adipose tissue; and other materials. Autologous materials are derived from tissues, cells or fluids harvested from the patient, and in many cases such autologous materials undergo varying degrees of processing to concentrate certain of their components, or to otherwise accentuate their healing potential, followed by their local re-administration to the same patient in regions of the body where tissue healing, repair, growth, regeneration, or augmentation are desired, or which otherwise warrant enhancement or alteration. Some of these autologous materials also have non-autologous versions (for example, allogeneic, xenogeneic, synthetic, or recombinant forms) that may be considered functional equivalents to certain autologous material counterparts.

The advantages of some autologous materials relate to their autologous nature, which is appealing to many patients and clinicians, in part because autologous products are biocompatible and carry few safety risks. Autologous materials are also appealing based on their capacity to deliver or induce the expression of various endogenous growth factors and other molecules, fluids, tissues, cells, or matrices involved in tissue growth, regeneration, and repair. Some autologous materials, including cell-based therapies, offer the potential to directly or indirectly reconstitute or enhance cellular repertoires or milieus within the body to promote healing through tissue remodeling, growth, repair, regeneration, augmentation, or reconstruction, either through actions performed by the administered cells and their progeny, or by interactions between the administered cells or their progeny and resident cells within the patient that participate in beneficial tissue responses. Other autologous materials, including platelet-rich or platelet-containing autologous materials, may confer therapeutic benefits via the release of platelet-derived growth factors that are captured, retained, and in some cases concentrated during their preparation, and/or are secreted by the re-injected platelets that are activated ex vivo or become activated upon interaction with resident tissues at the site of their re-administration. Some autologous materials offer the potential advantage of a gel-like consistency that derives from fibrin formation and fibrin cross-linking, attributes that may be exploited to arrest bleeding and promote wound closure, or to provide a degradable provisional matrix upon which cells can act to reconstitute tissue, or to provide a fibrin matrix that controls the release of endogenous growth factors or to bind together and thereby contain grafting materials in ways that minimize their undesirable leakage or migration from the graft recipient site.

Though widely utilized across numerous therapeutic areas, autologous materials have certain limitations as therapeutics, particularly with regard to inconsistent or inadequate efficacy. Substantial variations between patients is one potential source of inconsistent efficacy of autologous materials therapies; suboptimal patient-specific characteristics, for example those associated with advanced age, smoking, diabetes, or immune system disfunction, may lead to the production of suboptimal autologous materials, and to suboptimal responses to autologous materials therapies. Variations in the processing of autologous materials from their component materials is another potential source of their suboptimal or inconsistent benefits. Reasons for suboptimal and inconsistent efficacy with autologous materials is likely to be multifactorial, and current hypotheses explaining these limitations are uncompelling and often lacking clear scientific bases or rationale.

PRTs, as well as other platelet-containing therapies such as whole blood and plasma, have pro-healing attributes that derive from various growth factors, cytokines, and other proteins stored in platelet granules. These factors are released when platelets are activated, whether by the addition of exogenous coagulation factors ex vivo or in vivo, or by interactions with endogenous platelet-activating factors and conditions at the site where harvested platelets are re-applied. These growth factors include platelet-derived growth factor (PDGF), transforming growth factor beta (TGF-beta), vascular endothelial growth factor (VEGF), basic fibroblast growth factor (bFGF), insulin-like growth factors (IGFs), bone morphogenic proteins (BMPs), and others.

Substantial research and clinical applications exist for using some platelet-containing or platelet-rich therapies for the purpose of stimulating osteoblasts and augmenting local bone formation. Several conditions or states lead to systemic or local deficiencies in the mass, volume, density, and strength of bone (collectively referred to as “bone stock”). For example, some individuals attain suboptimal peak bone mass during their youth, leaving them vulnerable to the consequences of reduced bone stock during adulthood. Systemically low or suboptimal bone stock can also result from, or be exacerbated by increased bone loss after the menopause, or from hypogonadism, or from various secondary causes such as glucocorticoid therapy, or from idiopathic causes. These and other forms of reduced systemic or local bone stock leave regions of the skeleton vulnerable to fracture, and to suboptimal healing after skeletal injuries, and to suboptimal outcomes after various surgical procedures. Additionally, locally-suboptimal bone stock, which carries similar liabilities as would a systemic deficiency, can arise from congenital birth defects, inadequate bone growth, bone injury, malignancies, benign tumors, cysts, surgical procedures, local infection, host responses to local infection (e.g. periodontal disease), impaired bony healing, which may include insufficient chondrogenesis and/or osteogenesis, and other causes. Treatments that increase bone formation have the potential to mitigate these and other adverse consequences of inadequate bone stock.

Bone formation may also be desired to facilitate repair, reconstruction, reconstitution, or augmentation at specific skeletal sites even if initial bone stock is not necessarily suboptimal. For example, fracture healing involves the formation of new bone that bridges the fractured ends and restores bone continuity, shape, and function. Limb lengthening by distraction osteogenesis requires new bone formation to increase bone length. The repair of craniomaxillofacial defects often requires new bone formation to bridge and graft bones to reestablish skull, jaw, and/or facial structures and continuity. The regeneration of bone following tooth loss or tooth extraction requires new bone formation to facilitate the placement of dental implants. The repair of tendon or ligament injuries often involves the bony integration (osseointegration) of tendons, ligaments, or tendon-bone or ligament-bone grafts into the skeleton. In some cases, short- and long-term problems arise with bone tunnels created during ligament reconstruction that results in reduced local bone stock, such as bone tunnel enlargement or local osteolysis. In order to achieve the desired fusion of two bones across a joint space (arthrodesis), new bone formation is needed to achieve stable fixation of the fused construct, which may alleviate pain or otherwise preserve or restore physical function. In other cases, such as esthetic applications, a patient may desire esthetic augmentation that may be achieved by increasing bone formation in facial bones to enhance dermal projection at esthetically relevant sites such as the jaw line, chin, and cheek bones. This has been accomplished clinically by the sub-periosteal implantation of bone graft particles for a more durable esthetic effect compared with dermal fillers and plumping agents. Smokers and patients with diabetes can also benefit from agents that stimulate bone formation due to the adverse effects of smoking and diabetes on osteogenesis and/or chondrogenesis, which can impair bone healing.

Some studies show that locally-applied PRTs in various forms can increase local bone formation and regeneration, but in general, bone augmentation and regeneration with PRTs in orthopedic, spine, dental, craniofacial, and other therapeutic areas is modest at best, and inconsistent from study to study. The clinical use of PRTs for bone augmentation is frequently described as controversial due to the limited evidence for its efficacy and the lack of understanding of PRT characteristics that contribute to its insufficient or inconsistent bone-augmenting effects.

Existing therapeutic approaches to increasing bone formation in patients include systemically-administered parathyroid hormone 1 receptor (PTHR) agonists teriparatide and abaloparatide, which stimulate osteoblasts and increase bone formation throughout the skeleton. These agents were initially developed to increase bone mass and reduce fracture risk in patients with osteoporosis. The systemic administration of PTHR agonists to animals or humans can improve bone stock and enhance bone healing in certain settings. Animal studies show that systemic teriparatide (TPTD; PTH[1-34]) can improve the osseointegration of titanium implants, promote certain aspects of fracture healing, and promote alveolar bone gains in patients with periodontal disease. Systemic TPTD showed favorable effects on fracture healing and spinal fusion in some but not all clinical studies as an adjuvant. Nevertheless, PTHR agonist therapy for local bone augmentation has several limitations. Firstly, PTHR agonists are not approved for local bone augmentation, and their off-label use in such settings often requires out-of-pocket payment by patients, who must then self-inject themselves for several weeks with the hope of achieving unproven efficacy, which may limit compliance and persistence. Secondly, while surgeons are familiar and facile with local procedure-based delivery of therapeutic agents, they are less familiar with systemic administration of bone anabolic agents and other drugs, which often requires a prescription and conferral with a patient's primary doctor or other health care professionals or care-givers. Another limitation of PTHR agonist therapy is its tendency to increase bone resorption, which can limit its ability to improve bone stock systemically and locally. Furthermore, systemically-administered TPDT, abaloparatide, and most other bone anabolic therapies are expected to have effects on bone beyond the site of injury or deficiency, which may be undesirable for patients who otherwise have adequate systemic bone mass. TPTD and other bone anabolic agents delivered systemically might also require larger amounts of the drug or longer durations of treatment compared with locally-delivered drug in order to achieve a satisfactory local effect.

Another investigational approach to improving bone stock and bone healing involves activation or further stimulation of intracellular signaling induced by certain members of the wingless-related integration site (Wnt) family of secreted factors. Wnts are critical mediators of cell-to-cell signaling during embryonic development. Wnts also play important roles in tissue homeostasis and repair in post-natal animals, including humans. Wnt activity is regulated temporally and spatially via restricted expression of their cognate receptors, which include lipoprotein receptor-related proteins 5 or 6 (LRP5/6) and a co-receptor from the Frizzled (FZD) family. A variety of secreted inhibitory factors are also spatiotemporally regulated to provide regulatory control (inhibition) of Wnt signaling. One of the hallmarks of (canonical) Wnt signaling is the elevation of beta-catenin (β-catenin) in the cell cytoplasm, which leads to its accumulation in the nucleus and the formation of complexes with certain transcription factors that ultimately lead to changes in gene expression. Wnt signaling is one of the major pathways that promote bone formation, and Wnts are also potent mediators of skeletal development and bone accrual. Gain-of-function mutations in the Wnt receptor LRP5 leads to increased Wnt signaling and high bone mass in mice and in humans, whereas loss-of-function LRP5 mutations impair Wnt signaling and lead to low bone mass.

Various pharmacological means of directly activating or enhancing or potentiating Wnt signaling can lead to increased bone formation and improved bone stock. The psychiatric drug lithium chloride (LiCl) activates Wnt signaling by inhibiting the activity of glycogen synthase kinase-3 (GSK3), thereby preventing β-catenin degradation. Systemically administered LiCl increases bone mass in normal mice, and LiCl can also have positive effects on bone healing in rodents, though these effects may depend on the timing and duration of its administration. Wnt signaling can also be induced via the administration of various recombinant Wnts. For example, Wnt3a promoted the osteogenic differentiation of cultured bone marrow-derived mesenchymal stem cells (BM-MSCs), and local liposome-based administration of recombinant Wnt3a promoted bone healing in skeletal defects in mice. Wnt signaling can also be promoted by Wnt surrogate molecules that are engineered to bring together (i.e., heterodimerize) Wnt receptors (Frizzleds and LRP5/6) in ways that induce downstream signals. Another way of promoting Wnt signaling involves R-Spondins, which are extracellular ligands that promote Wnt signaling via binding to various leucine-rich repeat-containing G-protein-coupled receptors (LGRs) that may enhance the function of Wnt receptor complexes. The Wnt signaling pathway can also be stimulated by various extant and yet-to-be-discovered factors that work intracellularly to promote, mimic, or otherwise interact with intracellular regulators of Wnt signaling. Such intracellular regulators include but are not limited to β-catenin, axin, T-cell factor (TCF), lymphoid enhancer factor (LEF), deoxycholic acid (DCA), adenomatous polyposis (APC), Groucho, disheveled (DVL), protein phosphatase 2A (PP2A), and frequently re-arranged in advanced T-cell lymphomas (FRAT-1). Wnt signaling can also be increased by factors that promote the secretion of Wnts, a process that is governed in part by various biochemical processes such as Wnt palmitoylation and deacetylation.

Other approaches to promote Wnt signaling involve the targeted inhibition of endogenous extracellular Wnt antagonists and other Wnt pathway inhibitors including intracellular factors, collectively referred to hereafter as inhibitors of Wnt signaling. A variety of inhibitors of Wnt signaling exist, many of which are secreted or membrane-bound factors including sclerostin, Dickkopf-related protein 1 (DKK1), Wnt inhibitory factor 1 (WIF-1), Wise which is encoded by gene Wise/SOSTDC (Sclerostin Domain Containing 1 gene), Kremin 1 and Kremin 2, and secreted frizzled-related proteins (S-FRPs). These regulatory molecules are generally expressed under spatial and temporal controls in response to numerous biological and biomechanical cues. One well-validated inhibitor of Wnt signaling that limits bone formation is sclerostin, a secreted factor that acts by binding to and inhibiting the Wnt signaling potential of LRP5/6 in conjunction with its co-receptor Frizzled. Loss-of-function mutations in the gene encoding sclerostin leads to high bone mass in humans and animals, and systemic overexpression of sclerostin leads to low bone mass. Sclerostin expression is upregulated locally by osteocytes after skeletal injury, which may limit bone healing responses. Several therapeutic anti-sclerostin antibodies (Scl-Ab) have been shown to increase bone formation, bone mass, and bone strength throughout the skeleton. To the best of Applicant's knowledge, all clinical studies and almost all animal studies performed to date with Scl-Ab therapy involved systemic Scl-Ab administration. Systemically administered Scl-Ab therapy increased bone formation in women with postmenopausal osteoporosis, leading to increased bone mass and reduced fracture risk. Preclinical studies show that systemically administered Scl-Ab can improve bone healing in orthopedic models, including fracture healing, and in dental disease models, including periodontal bone loss and bone regeneration in the edentulous alveolar ridge.

Another systemic approach to increasing Wnt signaling and bone formation involves the targeting of Dickkopf-related protein 1 (DKK1), a secreted protein that inhibits canonical Wnt signaling. DKK1 is part of a repertoire of factors that provide tight spatial and temporal controls over Wnt signaling. Animal studies indicate that DKK1 expression by osteocytes is reactively upregulated after skeletal injury, which has been suggested to inhibit bone regeneration and bone healing. Systemic administration of antibodies that inhibit the activity of DKK1 (Antibodies that bind to human DKK1; DKAB) has been shown to promote bone healing and bone repair in animals, with the interesting and unique finding that systemic DKAB administration has minimal effects on bone formation or bone mass at uninjured skeletal sites.

In addition to their roles in bone, Wnt signaling and DKK1 play potentially important roles in the homeostasis, regeneration and repair of numerous soft tissues, including skin and hair. Wnt signaling appears to be involved in skin repair after injury, and cutaneous wounds express various Wnts during the early phase of healing, including Wnts 1, 3, 4, 5a, and 10b. Modulation of Wnt signaling has been implicated in the regulation of wound healing in mice. Wnt signaling may also regulate the vitality, survival, and regenerative potential of hair follicles. Wnt signals appear to be involved in hair follicle development, and activation of dermal Wnt signaling in animals increased hair follicle density.

Dermal wounds can occur in numerous manners, such as in trauma, following emergent or elective surgery, related to medical co-morbidity such as diabetic foot ulcers, excisional surgery such as in cancer, injections, allergic reactions, burns, dermatologic disorders, plastic surgery, maxillofacial reconstruction for acquired or congenital disorders or defects or any skin procedure or their complications relating to eschar, scar, healing or esthetic appearance or color. Current mechanical wound healing therapies include local devices such as sutures or tapes or adhesives or sealants that act mechanistically to bridge the wound or tissue gap, thereby allowing natural biological healing processes to proceed. In other cases, wound dressings with attached suction devices may augment healing and wound debridement by helping bridge the wound gap and control swelling and fluid exudate. The most common wound dressing is a bandage type of covering for wounds which provides a protective layer to cover the wound. The material for the bandage may be varied from simple fabric to hydrocolloid, hydrogel or other. Some wound healing products work as serial debridement. The use of allograft and xenograft sourced tissue has also been used for wounds in the form of layers or coverings. There are also pro-coagulant sealant products such as Tisseel, an allogeneic form of fibrin.

Various autologous materials including PRTs have also been studied as investigational therapies for promoting the healing of skin and dermal wounds, including pressure ulcers, venous stasis ulcers, burns, and surgical or cancer-related incisions. Some studies also investigated PRTs with the addition of various stem cell preparations or adipose-derived materials for wound healing. Mechanisms by which PRTs may promote wound healing are unclear, but it is commonly believed that enriched growth factors contribute to their beneficial effects. Overall, the benefits of PRTs and other autologous materials for skin and dermal wound healing in medical or esthetically-focused cases are limited and inconsistent. Thus, despite these various therapeutic options, many of which lack rigorous evidence for clinical efficacy, there remains an unmet need for methods, treatments, and systems for more effective wound healing.

Current approaches to hair restoration include hair grafting, whereby viable hair follicles are transplanted from one region of the scalp to other regions where additional hair is desired. This surgical approach is reasonably efficacious, but some follicles do not survive transplantation, and some do not generate hair shafts with optimal diameter or growth rate. Pharmacological treatments include topical minoxidil, an antihypertensive vasodilator medication that is indicated for the treatment of androgenic alopecia. Around 40% of men experience hair regrowth after 3-6 months of minoxidil therapy, though its use must be continued indefinitely to support the vitality of existing hair follicles and to promote hair regrowth. Minoxidil therapy appears to be less effective when the area of hair loss is large, and Minoxidil is only indicated for central (vertex) hair loss. Minoxidil therapy is also associated with several side effects, including temporary hair loss, burning and irritation of the eyes, itching, and unwanted hair growth elsewhere in the body. Another pharmacological treatment is oral finasteride, which acts by inhibiting the production of dihydrotestosterone (DHT), a hormone that plays an important role in the development of AGA. Finasteride therapy can slow further hair loss in men and cause some improvements to hair density, though finasteride does not have similar benefits in women, and its benefits in men can vary according to scalp region. Finasteride effects wane when the drug is discontinued, and it has several known side effects and safety risks, including sexual dysfunction. There remains a clear unmet need for more effective and safe hair restoration therapies.

PRTs and other autologous materials have also been studied as treatments for hair loss, including in patients with male pattern baldness (androgenic alopecia [AGA]), alopecia areata, and in female patients with hair loss. Local injections or topical applications of PRTs into or upon the scalp have been tested in patients desiring hair augmentation based on the premise that certain platelet-derived growth factors are involved in promoting hair thickness, hair density, hair growth, or hair regrowth. Many growth factors implicated in hair growth and hair follicle vitality, including PDGF, TGF-beta, IGF-I, FGF-2, and VEGF, are also enriched in PRTs, including PRP. Some published studies indicate that PRTs have the potential to reduce hair loss, increase hair shaft thickness, improve hair follicle density, and enhance the efficacy of hair grafts and other hair restoration therapies, such as minoxidil or finasteride therapy. However, the benefits of PRTs for hair restoration and hair augmentation are often limited, inconsistent, and short-lived, requiring continued re-applications to maintain efficacy that is usually modest at best. Current PRP therapies are unable to arrest or reverse the hair loss process, and there is limited evidence from blinded placebo-controlled clinical trials indicating that PRTs can meaningfully promote hair regrowth or reduce hair loss. Optimal platelet concentrations of PRTs used for hair restoration are unclear, and variations in preparation methods have been proposed as a source of inconsistency in terms of the efficacy of PRTs for hair preservation, restoration, and augmentation.

Despite the importance of Wnt signaling in tissue regeneration, investigators in the fields of regenerative medicine, orthopedics, wound care, and hair restoration are largely unaware that platelets store DKK1 in their alpha granules, and that this DKK1 is rapidly released when platelets become activated. This lack of awareness is reflected in the complete absence of published data on DKK1 levels in platelet-containing, platelet-enriched, or platelet growth-factor-enriched ABMs such as PRP, PRF, activated plasma, bone marrow aspirate, and reamer-irrigator aspirate. The lack of such inquiries may be surprising considering the intense and longstanding interest in deciphering the key elements and compositions that underlie the regenerative effects and limitations of these and other ABMs. Hundreds of publications report on the concentration and balance of platelet-released growth factors, various cytokines, cells, and other biological components that may favorably or unfavorably influence the regenerative milieu of PRP, bone marrow-derived ABMs, and other ABMs. Yet despite published evidence that DKK1 impairs bone formation, wound healing, and hair growth, which are conditions that are often treated with platelet-containing ABMs, there appears to be no recognition that DKK1 may be limiting the therapeutic potential of such ABMs, or that DKK1 inhibition may enhance the efficacy of such ABMs.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### SUMMARY OF THE INVENTION

This invention provides methods of promoting local bone formation, hair growth, or wound healing by administering a variety of autologous materials, hereby referred to as autologous body materials (ABMs) and their functional equivalents, in combination with an agent that promotes Wnt signaling, in which typically the cells within the ABM, or the resident cells at the target sites where the ABM or its functional equivalent is administered, have an insufficient level of Wnt signaling. The administration of agents that promote Wnt signaling to sites of injury, disease, or surgery, or that are otherwise in need of tissue regeneration or augmentation, in conjunction with ABMs, is conceived to enhance the regenerative milieu in ways that improve therapeutic responses. In some aspects the ABMs are prepared to have DKK1 removed or reduced before administration to a subject in lieu of adding an exogenous promoter of Wnt signaling to the ABM.

Various embodiments of the methods provide that an agent that promotes Wnt signaling is an inhibitor of DKK1. The disclosed methods include administering an inhibitor of DKK1 with an ABM to a subject for promoting local bone formation, hair growth, or wound healing. We propose that the modest, absent, or inconsistent therapeutic benefits of platelet-containing and platelet-enriched ABMs in those therapeutic settings relates to abundant platelet-released DKK1. The addition of a pharmacological inhibitor of DKK1 (i.e., an anti-DKK1 agent, such as DKAB) to PRTs would block the activity of much of the DKK1 within the PRT, thereby promoting Wnt signaling.

Further embodiments provide that agents that promote Wnt signaling by means other than DKK1 inhibition (e.g., LiCl) could be added to ABMs as a way of overcoming (i.e., compensating for) the adverse effects of high DKK1 levels, thereby restoring or otherwise increasing local Wnt signaling to enhance therapeutic responses. The disclosed methods further include preparing ABMs to reduce their content of DKK1 prior to administering the ABM to the patient, as an alternative to adding an exogenous promoter of Wnt signaling. A reduction in DKK1 levels is accomplished by contact between the ABMs and one or more agents that function by directly binding to DKK1 resulting in physically separating DKK1 from PRTs and other ABMs. For example, the disclosed method includes using an apparatus to strip the ABM of much of its soluble DKK1. With that approach, the ABM can be delivered to the patient without its DKK1, and with minimal patient exposure to the anti-DKK1 agent, because the anti-DKK1 agent remains within or is otherwise associated with the PRT preparation system. Alternatively, a method includes administering an ABM in combination with an anti-DKK1 agent to a subject, which results in a therapy that has minimal bioactive DKK1 within it and also inhibits the activity of endogenous DKK1 found at the site of administration, e.g., endogenous DKK1 produced by local platelets, osteocytes, or other cells in the local target tissue.

Hematoma formation is common after fracture, and hematoma also forms during distraction osteogenesis procedures. Hematoma contains several platelet-derived growth factors that are widely believed to promote healing, and yet the ability of DKAB to further promote fracture healing when administered to animals with fresh fractures indicates that despite the likely presence of various growth factors released by hematoma-associated platelets and other cells, the biochemical milieu at fracture sites is not optimized for the rapid healing and biomechanical restoration of fractured or distracted bones. A major limitation of distraction osteogenesis is the prolonged time patients must remain in a cumbersome external fixator while new bone forms and consolidates. DKK1 is not expressed during the post-osteotomy latency (i.e. resting) phase of DO, but DKK1 expression is upregulated during the distraction and consolidation phases of the procedure, which may control (i.e. limit) the rate and extent of new bone formation. The local application of DKAB combined with platelet-containing ABMs has the potential to promote Wnt signaling and thereby increase bone formation without impairing the ability of the various platelet-secreted growth factors from the ABM and the osteotomy-induced hematoma to promote healing via their individual or combined mechanisms. Further aspect of this invention provides administering anti-DKK1 agents such as neutralizing anti-DKK1 antibodies (DKAB) to a subject to promote bone augmentation at sites of skeletal injury or surgery, wherein the uninjured skeletal sites have little to no correlation to DKK1 that is released by activated platelet in response to local bone bleeding, injury, or surgery. Platelet activation at the site of bone injury, damage, or surgery creates a local increase in DKK1 that inhibits the rate and degree of local bone formation. Alternatively, other agents that directly or indirectly promote Wnt signaling could be used in lieu of or in addition to an anti-DKK1 agent to overcome the deleterious effects of high local DKK1 levels released from activated platelets.

In some embodiments, other promoters of Wnt signaling (e.g., a Wnt surrogate that heterodimerizes Wnt co-receptors) in lieu of or in combination with anti-DKK1 agents are mixed with ABMs and delivered to a distraction osteogenesis (DO) site as a way of overcoming or compensating for the adverse effects of high DKK1 levels within the ABM and within the distracted site itself.

Also provided are compositions and methods for restoring or augmenting Wnt signaling, thereby promoting local bone formation, hair growth, or wound healing, which include a system of an agent that promotes Wnt signaling (e.g., an anti-DKK1 antibody) in combination with a non-platelet-containing ABM such as stem cells. Exemplary and non-limiting stem cell-based ABMs include bone marrow aspirates and reamer-irrigation aspirates, which will include platelets that are likely to undergo activation and DKK1 release as the ABM is harvested and prepared.

Another embodiment of the method provides administering a promoter of Wnt signaling in an autologous fibrin-based ABM to a subject for promoting local bone formation, hair growth, or wound healing. Fibrin gel, and PRF, have a gel-like form factor imparted by fibrin formation and cross-linking, creating a matrix with several potential therapeutic benefits. These gel-like matrices have the potential to bind tissues together to arrest bleeding and promote wound approximation and healing, and may also provide a degradable provisional matrix upon which resident cells can act to reconstitute tissue. The cohesive properties of some gel-based ABMs can also be exploited to help bind and contain grafting materials, thereby improving their physical handling characteristics and limiting their undesirable leakage or migration from the graft recipient site. Gel-based ABMs can also confer sustained release of endogenous growth factors they may contain, as well as of therapeutic agents that promote Wnt signaling. Considering that autologous fibrin-based ABMs are often used in dentistry, maxillofacial surgery, orthopedic surgery, plastic surgery, and aesthetics, and considering that DKK1 secretion from platelets may exerts untoward effects at the very sites where autologous fibrin-based ABMs are often applied, there is a unique and therapeutically rational opportunity to add an anti-DKK1 agent or other promoter of Wnt signaling to fibrin-based ABMs in certain therapeutic settings. One aspect of this embodiment has the potential to immediately improve the biochemical milieu at the delivery site in ways that favor healing, either by neutralizing DKK1 within the ABM or site of its administration, or by compensating for high DKK1 by promoting Wnt signaling through other means that override DKK1 inhibition. The local delivery of a promoter of Wnt signaling in an autologous fibrin-based ABM has the potential to immediately improve the biochemical milieu at the delivery site in ways that favor healing, This approach may lead to better efficacy compared with that which would be achieved by injecting the therapeutic agent by a standard subcutaneous route at a site that may be distant from the injury or surgery site, especially with large-molecule therapeutic agents like antibodies, which can take up to a week or more to achieve peak blood levels. Platelet release of DKK1 is likely to be an acute phenomenon that may begin to wane by the time peak drug levels are achieved after a subcutaneous injection, which would allow relatively more local DKK1 to inhibit Wnt signaling and limit healing compared with the more immediate effects achieved by physically placing the agent directly upon or within the site of injury, treatment, or surgery.

A system or composition is provided including an autologous body material (ABM) and an inhibitor of Dickkopf-related protein 1 (DKK1). In various embodiments, the ABM is platelet rich, platelet-containing, or has platelet-derived factors. Exemplary ABMs include but are not limited to PRTs such as platelet-rich plasma (PRP; unclotted and clotted forms), and platelet-rich fibrin, PRF, platelet gel, as well as platelets, plasma, bone autografts, bone allografts, surgical bone, surgical blood, peripheral blood, reamer-irrigator aspirate, bone marrow and bone marrow aspirate concentrate (BMAC), and any combinations thereof.

In various embodiments, a promoter of Wnt signaling pathway is an inhibitor of Dickkopf-related protein 1 (DKK1), or of sclerostin, or of Wnt inhibitory factor-1 (WIF-1), or of Kremens, or of Frizzled function, or of secreted frizzled-related proteins (SFRPs), or of Wise/SOSTDC, or of other factors that antagonize or otherwise inhibit Wnt signaling. Those skilled in the art would also recognize that other agents that promote Wnt signaling, including certain Wnts (e.g. recombinant forms including those that may be formulated in liposomes), agents that promote Wnt secretion, lithium chloride, R-Spondins, and Wnt surrogates that heterodimerize Wnt co-receptors also have the potential to improve the regenerative milieu of various ABMs, especially those ABMs with elevated DKK1 levels, where the promoter of Wnt signaling will overcome or compensate for the Wnt-inhibiting effects of the elevated DKK1 in the ABM and in the subject where the ABM is administered.

Various forms of an anti-DKK1 agents or other promoters of Wnt signaling pathway and how they are delivered are provided. In some embodiments, an anti-DKK1 agent is combined with an allogeneic material prior to incorporation or encapsulation in an ABM. For example, a lyophilized form of an anti-DKK1 agent (e.g., DKAB) or other promoter of Wnt signaling is mixed with or applied to an allogeneic bone graft product, followed by their combining with an ABM prior to administration to a subject. In another embodiment, an anti-DKK1 agent or other promoter of Wnt signaling is prepared with an allogeneic form of a platelet-rich therapy, or an allogeneic bone marrow-based therapy, or an allogeneic stromal vascular fraction-based material prior to combination with an ABM. For example, an allogeneic platelet-rich therapy is produced from a pooled source of human blood, which is then combined with an anti-DKK1 agent or other promoter of Wnt signaling to produce an allogeneic material plus therapeutic complex, which is subsequently combined with an autologous material (i.e. ABM) of a similar or in most cases a different autologous tissue type (for example, autologous stem cells) at the time of administration (e.g., on the surgical field).

In another embodiment, an ABM is a material in or upon the body of a subject, such as blood or other fluids that are locally expressed during trauma, decortication, endplate preparation, microfracture surgery or microneedling procedures, to which and an anti-DKK1 agent or other promoter of Wnt signaling can be added without necessarily harvesting said blood or other fluids or tissues from the patient for later re-administration. In some scenarios it would be advantageous to supplement an anti-DKK1 agent or other promoter of Wnt signaling with thrombin or other agents that activate platelets, followed by their co-injection into an ABM that remains in situ, such as a fracture hematoma. Such an approach may induce platelet release of additional growth factors, along with DKK1 that can be readily inhibited by the anti-DKK1 agent or compensated for by the added promoter of Wnt signaling.

In another embodiment, an anti-DKK1 agent or other promoter of Wnt signaling is combined with an allogeneic material such as a bone graft substitute, with or without another ABM, and administered to the subject. Yet another embodiment provides that an anti-DKK1 agent or other promoter of Wnt signaling pathway is mixed or associated with purified or recombinantly-produced body materials, such as recombinant or purified fibrinogen, fibrin, fibrin matrix, fibrin glue, or fibrin gel.

In some embodiments, the composition is a plasma-based or PRP-based fibrin gel or another ABM with a clot-like consistency and which encapsulates or otherwise incorporates an anti-DKK1 agent or other promoter of Wnt signaling, such that the agent is released in a slower and more sustained manner after local administration compared with the same agent administered without said gel or ABM. The gel- or clot-like consistency of some ABMs is conferred by fibrin formation and/or fibrin cross-linking, but other ABMs including adipose and bone marrow can exhibit gel-like or matrix-like consistencies created by their intrinsic collagen-based reticular fiber networks. Fibrin and reticular networks may also provide a provisional matrix that supports angiogenesis and other tissue regenerative responses, and may also confer sustained release of various platelet-released growth factors that become trapped within it, as a function of the ABM's gradual fibrinolytic or collagenolytic degradation. Exemplary ABMs having a gel-like or clot-like consistency include clotted (i.e., activated) platelet-rich or platelet-containing therapies, such as clotted PRP, activated PRP, PRF, activated platelet-poor plasma (APPP), clotted forms of bone marrow aspirate (BMA), bone marrow cells (BMCs), BMA concentrate (BMAC), clotted surgical blood, clotted peripheral blood, clotted reamer-irrigator aspirate, activated plasma (i.e., plasma gel), and adipose derived stromal vascular fractions (AD-SVF) such as adipose derived ABM, and any combinations thereof. The gel-like or clot-like consistency of fibrinogen-containing ABMs may be induced in vivo or ex vivo through exposure to endogenous or exogenous factors that activate platelets or promote fibrin formation and/or cross-linking (e.g. thrombin, collagen, tissue factor). These favorable consistencies may also be achieved ex vivo by exposure to other agents that activate platelets or promote fibrin formation, such as silica particles coating the inside of blood collection tubes or other vessels or transfer devices.

A process of promoting local bone formation or increasing local bone volume or local bone density, i.e., improved local bone stock, is provided including administering a composition containing an ABM and an anti-DKK1 agent or other promoter of Wnt signaling to a subject in need thereof. In some aspects, the composition is administered locally at a site that is platelet rich or that has platelet-derived factors. Exemplary platelet-derived factors include PDGF, TGF-beta, VEGF, bFGF, and IGFs.

Various embodiments provide that the process of administering the composition containing an ABM and an anti-DKK1 agent leads to reduced levels of bioactive DKK1 within the ABM, or within the site where the ABM is administered, or both, thereby alleviating DKK1-mediated inhibition of bone formation, wound healing, or hair growth while maintaining other healing properties of the ABM. Platelets associated with the administered ABM may continue to release bioactive DKK1 after the ABM has been delivered, and the composition containing an anti-DKK1 agent in an ABM allows for ongoing neutralization of bioactive DKK1 within the patient after the ABM is applied. In other aspects, resident osteocytes and newly-arriving platelets, as well as extracellular fluid, are likely to contain or produce additional DKK1 beyond that which may be found in the ABM itself; and the composition containing an anti-DKK1 agent in an ABM allows for neutralization of this additional DKK1. As a corollary, various embodiments may include the composition of an ABM with agents that promote Wnt signaling by means other than the relief of DKK1 inhibition, whereby the agent overcomes or otherwise compensates for excessive DKK1 levels to foster Wnt signaling.

In some embodiments, a process is provided for administering cell-based ABMs encapsulating or incorporating an anti-DKK1 agent or other promoter of Wnt signaling to a subject in need thereof, where the ABM contains uncommitted stem cells with the potential to differentiate along the osteoblast lineage. This process includes administering cell-based ABMs that can be BMA, bone marrow stromal vascular fraction (BM-SVF), BMCs, BMACs, mesenchymal stem cells, adipose-derived tissue, micronized adipose tissue (MAT) or enzymatically digesting adipose derived stromal vascular fraction (AD-SVF), bone autograft, surgical blood, peripheral blood, reamer-irrigator aspirate (MA), and any combinations thereof. The aforementioned agents will induce a greater rate and/or proportion or survival or proliferation of stem cells undergoing osteogenic differentiation by promoting Wnt signaling within the ABM prior to its administration, and/or within the site of administration where the balance of endogenous Wnt promoters and Wnt inhibitors may be suboptimal, in many cases due to the influence of platelet-released DKK1 induced by injury or surgery.

In another embodiment, the system and method include a process that uses an exogenous agent that physically removes or strips DKK1, or other inhibitors of Wnt signaling, from the ABM prior to the ABM's administration to the patient. In this manner, the ABM is delivered to bone, damaged tissue, scalp, skin or other bodily regions with minimal or no amount of the DKK1 or other inhibitor of Wnt signaling, or any such exogenous agent entering the body. The removal of DKK1 or other inhibitor of Wnt signaling may occur prior to preparation of the ABM, or during preparation of the ABM, or after preparation of the ABM by using a novel device to extract the anti-DKK1 agent or other soluble inhibitor of Wnt signaling. By this ‘stripping’ process, the ABM remains enriched in all the various GFs, cytokines, and other beneficial factors from platelets or serum or cells or stroma, but with reduced levels of DKK1 or other inhibitor of Wnt signaling. The ‘stripping’ may be accomplished through the development of a novel apparatus that includes a coating or formulation or mesh or beads or structure or sieve within the apparatus that binds, or filters, adheres, or otherwise eliminates or destroys or renders inactive most of the ABM-related DKK1, or other inhibitors of Wnt signaling such as sclerostin, or SFRPs, or WIF-1, or Wise/SOSTDC, or multiple inhibitors of Wnt signaling, prior to administration of the ABM to the patient. In this manner, a biologically meaningful amount or proportion of the DKK1 or other inhibitor of Wnt signaling in the original autologous material, or in a partially or fully-processed ABM is removed, for example via the binding of autologous DKK1 to an immobilized DKAB or other anti-DKK1 agent. For example, DKAB-coated magnetic beads may be added to the ABM and then retained via magnetic forces while the ABM is recovered without said beads, to which the captured DKK1 remains bound. The reagents and equipment used to bind and retain the autologous DKK1, or another soluble inhibitor of Wnt signaling, or multiple soluble inhibitors of Wnt signaling, are intended to remain within or around the apparatus used to separate the inhibitor or inhibitors of Wnt signaling from the ABM and are not intended to be administered to the patient.

Exemplary conditions to be treated by administering the composition or the system include implant fixation, fracture repair, arthrodesis, extraction socket preservation, alveolar ridge augmentation, spinal fusion, bone healing, tendon or ligament reconstruction in bone, distraction osteogenesis, esthetic appearance and facial bone geometry, congenital or tumor-induced bone deficiencies, bone cavity defects, bone cysts, and traumatic bone loss. Additional conditions to be treated by administering the composition or system include wound healing, chronic or otherwise, skin regeneration and hair growth. In some embodiments, the disease or condition to be treated, or the soft tissue damage to be repaired, comprises burns, scars, fistulas and general tissue loss; or hair loss.

The composition and process of treating a wound, or hair loss, or bone-related disease or disorder described herein directly or indirectly neutralizes or mitigates the Wnt-inhibiting effects of DKK1 via the addition of an anti-DKK1 agent such as DKAB, or another promoter of Wnt signaling, to ABMs. The inclusion of an anti-DKK1 agent or other promoter of Wnt signaling causes little or no reductions in other endogenous components of the ABM that may induce favorable regenerative responses, while simultaneously promoting new bone formation, or improved or faster healing responses, or enhanced wound healing, or improved hair esthetics, by increasing Wnt signaling. In some cases, the addition of the anti-DKK1 agent to the ABM confers therapeutic benefits based not only on the neutralization of DKK1 within the ABM but also of local DKK1 found within the patient at the site where the ABM is applied. Similarly, the addition of other promoters of Wnt signaling besides DKK1 inhibitors to ABMs will also restore or augment Wnt signaling not only within cells found within the ABM itself but also within resident cells found at the site where the ABM is administered.

Other features and advantages of the invention will become apparent from the following detailed description, taken in conjunction with the accompanying drawings, which illustrate, by way of example, various features of embodiments of the invention.

### BRIEF DESCRIPTION OF THE DRAWINGS

FIG. 1 depicts ELISA-determined concentrations of DKK1, VEGF and PDGF in normal human plasma and serum from four healthy human donors. Plasma was prepared with blood tubes that contained sodium heparin, an anticoagulant that inhibits platelet activation, while serum was prepared from whole blood that was allowed to coagulate, a process that involves platelet activation. Concentrations of DKK1, VEGF, and PDGF are several-fold higher in serum compared with plasma, consistent with the release of each of those factors from activated platelets.

FIG. 2A depicts an increase in platelet counts for human platelet-rich plasma (PRP) versus normal plasma or platelet-poor plasma (PPP), and for normal plasma versus PPP. FIGS. 2B-2D depict the concentrations of soluble (i.e., extracellular) DKK1 (FIG. 2B), VEGF (FIG. 2C), and PDGF (FIG. 2D) determined by ELISA, which were significantly higher in plasma versus PPP, and in PRP versus plasma or PPP, in the absence of platelet activation with exogenous thrombin, calcium chloride, or other agents.

FIGS. 3A-3C depict the effects of calcium chloride, thrombin, or both on soluble (i.e., extracellular) concentrations of DKK1 (FIG. 3A), VEGF (FIG. 3B), and PDGF (FIG. 3C) in normal human plasma and PRP determined by ELISA. The addition of calcium chloride and thrombin to plasma or PRP at 37° C. for 30 minutes led to marked increases in DKK1, VEGF, and PDGF, consistent with their secretion from activated platelets. The modest effects of calcium chloride alone may relate to the method of plasma preparation, which relied on sodium heparin, an agent that prevents coagulation by mechanisms unrelated to calcium chelation.

FIG. 4 depicts the levels of soluble (extracellular) DKK1, VEGF, and PDGF in 1 mL samples of normal human serum incubated in the absence or presence of 2 million magnetic beads coated with DKAB. After a 70-minute incubation period, the beads were retained with a magnet while the bead-free serum was harvested and used in ELISAs to measure the various analytes. Incubation of serum with DKAB beads reduced DKK1 levels by around 85%, while causing only modest reductions in the levels of VEGF and PDGF.

FIG. 5 depicts the effects of 500,000 DKAB-coated magnetic beads on soluble (extracellular) DKK1 levels in 0.5 mL of normal human serum over time. After 15, 30, or 60 minutes of incubation, beads were retained with a magnet and bead-free serum was collected and analyzed for DKK1 levels by ELISA. Depletion of DKK1 from serum was significant with 15 minutes of bead incubation, and DKK1 was further depleted with 30 and 60 minutes of bead incubation.

FIG. 6 depicts the effects of 60 minutes of incubation with increasing numbers of magnetic beads coated with anti-human DKK1 antibody (DKAB) on levels of soluble (extracellular) DKK1 levels in normal human serum. After incubation, beads were retained with a magnet and bead-free serum was collected and analyzed for DKK1 levels by ELISA. Significantly greater DKK1 depletion was observed with increasing numbers of DKAB-coated beads.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, N.Y. 2006); March, Advanced Organic Chemistry Reactions, Mechanisms and Structure 7th ed., J. Wiley & Sons (New York, N.Y. 2013); and Sambrook and Russel, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application. For references on how to prepare antibodies, see D. Lane, Antibodies: A Laboratory Manual 2^{nd} ed. (Cold Spring Harbor Press, Cold Spring Harbor N.Y., 2013); Kohler and Milstein, (1976) Eur. J. Immunol. 6: 511; Queen et al. U.S. Pat. No. 5,585,089; and Riechmann et al., Nature 332: 323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. September; 23(9):1126-36).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Inhibitors of Wnt signaling” refers to a variety of endogenous molecules or substances that decrease the levels or function of various extracellular, membrane-localized, or intracellular proteins, lipids, or other molecules that are directly or indirectly involved in the initiation, propagation, or persistence of signals transduced by the functional binding of Wnts to their cognate receptors, primarily Frizzled family members, LRP5 or LRP6, or that endogenous molecules that inhibit R-Spondin receptors such as LGR4, LGR5, and LGR6 that potentiate Wnt signaling. Exemplary inhibitors of Wnt signaling include DKK1, sclerostin, WIF-1, SFRPs, FrzB, porcupine, notum/wingful, kremen 1, kremen 2, CTGF, axin, and APC.

“Anti-DKK1 agent” refers to therapeutic molecules that promote Wnt signaling by limiting the activity, or expression, or secretion, or bioavailability, or biological function of DKK1. Anti-DKK1 agents include those that directly bind to DKK1 in a manner that impairs the ability of DKK1 to attach to Wnt receptors in an antagonistic manner that reduces Wnt signaling, including but not limited to various therapeutic antibodies that bind and neutralize DKK1. Anti-DKK1 agents may also act by inhibiting the synthesis, expression, or secretion of DKK1, which can take the form of small molecules, or gene or virus vectors, or oligonucleotides, or other agents that prevent DKK1 gene transcription, or translation, or DKK1 protein synthesis, or DKK secretion in a soluble and functional form. Alternatively, or in addition, anti-DKK1 agents may act by competitively binding to Wnt receptors in ways that prevent or displace DKK1 binding, such that the anti-DKK1 agent itself or an endogenous or exogenous Wnt can promote or induce Wnt signaling, for example by functionally heterodimerizing Wnt receptors, or by acting in a dominant negative manner or as a decoy molecule or competitive antagonist that limits functional interactions between DKK1 and Wnt receptors. In some aspect, an anti-DKK1 agent is an anti-DKK1 antibody, which includes but is not limited to any one described in US Application Publication No. 20130209475 and in U.S. Pat. No. 8,338,576, which are incorporated by reference in their entirety, in an exemplary form of a free soluble antibody or an antibody that is found to a bead, a column or another vehicle.

“Promoter of Wnt signaling” or “agent that promotes Wnt signaling” refers to various endogenous or exogenous molecules or substances that are directly or indirectly involved in the initiation, propagation, persistence, amplification, or potentiation of signals that are initiated by or downstream from the functional binding of Wnts to their receptors, primarily LRP5 or LRP6 and various Frizzled family members. These events typically culminate in the accumulation of beta-catenin in the cell nucleus, leading to the transcription of genes involved in tissue growth or repair or augmentation responses. In addition to this ‘canonical’ signaling cascade, “promoters of Wnt signaling” also encompasses ‘non-canonical’ signaling, which is also triggered by Wnts and which involves signals mediated by intracellular calcium and JNK; non-canonical Wnt signaling is involved in the maintenance of adult stem cells as well as other traits that may be relevant to tissue growth, repair, or augmentation. Exemplary promoters of Wnt signaling include various Wnts that activate Wnt signaling, including but not limited to Wnt1, Wnt2, Wnt3, Wnt3A, and Wnt10B; or Wnt surrogates including VSD-LRP5/LPRP6 heterodimerizers such as scFv-DKK1c, which activate Wnt signaling by inducing heterodimerization of FZD-LRP5/6 co-receptors (Janda C Y, et al. Nature 2017; 545:234-237); or agents that neutralize or otherwise inhibit secreted inhibitors of Wnt signaling, including antibodies or other inhibitory proteins or molecules directed against sFRPs, WIF-1, Wise/SOSTDC, DKK1 (e.g. RH2-18, RH2-31, RH2-59, RH2-80, 11H10, 5.25.1, BHQ880, PF-04840082, JC18, HuMabCJ18, PF-04840082/RN-564, LSN2812176/DKN-01, and 4E4hum 7), sclerostin (e.g. romosozumab, AMG 167, blosozumab, BPS-804); or both DKK1 and another target such as sclerostin (e.g. Hetero-DS; Florio M, et al. Nat Commun 2016; 7:11505), or RANKL, or other therapeutic target molecules; or DKK1-based or DKK1-related oligopeptides that inhibit DKK1-LRP5/6 interactions (Park B M, et al. Yonsei Med J 2017; 58:505-513); or Wnt-FZD chimeras (Wyeth patent 20070072238); or RSpondin-1, RSpondin-2, and other ligands that activate LGRs in ways that enhance and potentiate the function of Wnt receptor complexes; or lithium chloride, lithium carbonate, and other agents that stabilize beta-catenin by inhibiting GSK-3β, including small molecules such as SB-216763 (Coghlan M P, et al. Chem Biol 2000; 7:793-803), BIO(6-bromoindirubin-3′-oxime) (Sato N, et al. Nat Med 2004; 10:55-63), and LY2090314 (Atkinson J M, et al. PLoS One 2015; 10:e0125028); or deoxycholic acid, which phosphorylates and stabilizes beta-catenin (Pai R, et al. Mol Biol Cell 2004; 15:2156-63); or pharmacological inhibitors of Axin, APC, Ck1a, groucho (Cavallo R A, Nature 1998; 395:604-8), Kremin-1, and Kremin-2; or activators of T-cell factor (TCF) and Lymphoid Enhancer Factor (LEF); or Porcupine, agonistic versions of Porcupine, and activators of Porcupine expression as well as other agents that palmitoleoylate Wnts to promote their trafficking and signaling; or agents that inhibit the de-acylation of Wnts, for example inhibitors of Notum (Wingful) (Suciu R M, et al. ACS Med Chem Lett 2018; 9:563-8); or Norrin, which promotes Wnt signaling by binding to FZD4 (Xu Q, et al. Cell 2004; 116:883-95); or inhibitors of sFRP1 such as WAY-316606 (Bodine P V, et al. Bone 2009; 44:1063-8); or activators of serine/threonine phosphatase PP2A, such as IQ1 (Miyabayashi T, et al. Proc Natl Acad Sci USA 2007; 104:5668-73); or activators of ARFGAP1 such as QS11 (Zhang Q, et al. Proc Natl Acad Sci USA 2007; 104; 7444-8); or AMBMP (2-amino-4-[3,4-(methylenedioxy) benzyl-amino]-6-(3-methoxyphenyl) pyrimidine), which promotes Wnt signaling through an unclear mechanism (Kuncewitch M, et al. J Trauma Acute Care Surg 2015; 78:793-800).

“Autologous body material” (“ABM”) refers to materials prepared from autologous sources, i.e., cells or tissues obtained from the same individual to whom said bodily materials will be re-administered or will otherwise be augmented in situ without first being harvested. Exemplary ABMs include but are not limited to platelet-rich therapies (PRTs) including platelet-rich plasma (PRP), platelet-rich fibrin (PRF), platelet and leukocyte-rich plasma and fibrin (L-PRP and L-PRF, respectively), platelets and platelet gel; fibrin gel; plasma; serum and hyperacute serum; whole blood, including peripheral blood and surgical blood that is or is not harvested (removed) from the patient before the addition of an anti-DKK1 agent or other promoter of Wnt signaling; various pluripotent stem cells including mesenchymal stem cells (MSCs) and other cellular fractions from various tissue sources including bone marrow and adipose; reamer-irrigator aspirate (MA); hematoma; and other materials. In many cases, ABMs will have allogeneic, xenogeneic, recombinant, or synthetic versions that are considered functionally equivalent to an ABM counterpart and are also included in this invention to the extent that adding an anti-DKK1 agent or other promoter of Wnt signaling would be expected to improve therapeutic benefits of said ABM.

“Platelet-Rich Plasma” (PRP) refers to blood plasma that has been enriched in platelets. It has also been found to be enriched in growth factors. Various forms of PRP can be made based on the system and protocol used to produce them. In 2009 Dohan Ehrenfest et al. produced a classification of 4 main families of preparation; 1) Pure PRP or Leucocyte-poor PRP, preparation without leucocytes and low-density fibrin network, 2) Leucocyte and PRP, preparation containing leucocytes and low density fibrin network, 3) Pure PRF of leucocyte-poor PRF, preparation without leucocytes and with high-density fibrin network, 4) Leucocyte-rich fibrin and PRF, preparations with leucocytes and with high-density fibrin network. Several authors conducted comparison studies of various PRP systems, the results of which indicate a substantial variation between each of these systems in centrifuge force, spin time, single vs dual spin, type of anticoagulant, and whether it is a buffy coat vs plasma-based system. Duraht et. al. further categorized PRP systems into “high concentration”, those with 5-9 times baseline platelet counts (concentrations over 750,000 per microliter) and “low concentration”, those with 2-3 times baseline platelet counts (concentrations around 200,000 per microliter). Mazzucco et al. defined the therapeutic value of PRP to be concentrations greater than 200,000 per microliter, thus both low and high concentrations have therapeutic value depending on the application. “Platelet-Poor Plasma” (PPP) refers to blood plasma with low platelet concentrations (<10,000 per microliter), though in many contexts PPP is simply the residual plasma that remains after a platelet-enriched fraction has been removed, which will typically involve a platelet count lower than that of normal unfractionated plasma. PPP typically have elevated levels of fibrinogen which make it advantageous in forming a fibrin-rich clot upon activation.

“Allogeneic material” refers to tissues or cells that are sourced from an individual other than the one to whom the material is ultimately delivered. Allogeneic materials may be processed in various ways to improve their biocompatibility. Exemplary allogeneic material includes but is not limited to an allograft bone product, an allogeneic platelet-rich or platelet-containing therapy produced from a pooled source of human blood, or a recombinantly produced or purified allogeneic fibrinogen or fibrin.

“Bone graft materials” refers to bone autografts (grafts from the patient's own bone stock), allografts (grafts from cadaveric bone stock), and synthetic bone graft substitutes (ceramics or demineralized bone matrix or composite materials). Graft materials are used to promote new bone formation and bone healing and to provide a substrate and scaffold for the development of bone structure. Bone graft materials also foster space maintenance to encourage bone growth over soft tissue infiltration. Bone graft materials can also be used to in combination to enhance or expand autograft materials in cases where the amount or volume of bone autograft is in limited supply or to reduce the degree of morbidity by minimizing the amount of bone autograft harvested.

“Device” refers to any forms of medical device intended for temporary or permanent implantation or fixation or insertion or application of any form in the body. Examples of device include implants, screws, nails, plates, rods, washers, anchors, buttons, pegs, pins, wires, fibers, sutures, adhesives, cements, demineralized bone matrix, bone chips, bone allograft, tendon allograft. In addition, it may involve the coating surface of an implant such as hyaluronic acid or hydroxyapatite coating. These devices maybe solid or porous or be made of any three-dimensional design. Other device examples include bone graft substitutes, prosthesis, stems, cages, mesh, sponges, beads, granules, tapes, strips, wound coverings (dermal, basement membrane, fascia, collagen, synthetic soft tissue, reconstituted soft tissue), tissue extenders (dermal, basement membrane, fascia, collagen, other minimally manipulated soft tissue, synthetic soft tissue, reconstituted soft tissue), capsule or tissue augmentation devices (dermal, basement membrane, fascia, collagen, other minimally manipulated soft tissue, synthetic soft tissue, reconstituted soft tissue).

“Defect or deficiency” refers to any defect, deficiency, discontinuity or void in a bodily tissue that warrants treatment or augmentation due to anatomical or physical or aesthetic limitations or surgical interventions or disease condition or injury. This may include bone reconstitution/augmentation/regeneration, wound healing, hair growth or hair restoration.

“Locally expressed autologous body materials” refers to ABMs that remain within a patient without being physically removed or otherwise harvested for processing or for combining with a promoter of Wnt signaling outside of the body. Locally expressed ABMs comprise autologous cells or tissues with some inherent therapeutic potential, including that which may be conferred by endogenous growth factors, endogenous pluripotent stem cells, or provisional matrix, to which a promoter of Wnt signaling may be directly administered. In some embodiments, locally expressed ABMs are present at or near the region of the body where tissue regeneration (bone, skin, or hair) is desired. In some embodiments, locally expressed ABMs include surgical blood, bone marrow, fracture hematoma, or extravasated blood that is expressed via micro-needling procedures or micro-fracture surgery or vertebral endplate preparation or bone decortication or osteotomy or other surgical procedures that create bleeding bone. In some embodiments, locally expressed ABMs exclude intravascular blood, subcutaneous adipose tissue, and ABMs that are distant from the site of treatment.

“Local bone reconstitution/augmentation/regeneration” refers to the formation of bone in a target tissue of interest. This may include restoration of lost host bone, or creation of new bone, extending to or beyond usual or expected anatomy.

“Wound healing” refers to the formation or remodeling or repair of damaged tissue whether epidermal, cutaneous, subcutaneous, dermal, hypodermal, endothelial, epithelial, or gingival, resulting in restored continuity and protection of bodily surfaces both internal and external. This may include repair, regeneration or rejuvenation of lost or damaged tissue extending to or beyond usual or expected anatomy, where caused by burns, cuts, punctures, abrasions, dehiscence, inadequate blood supply, pressure, friction, diabetes

“Hair growth” refers to the formation of hair whether bulbs, follicles or shafts. This may include repair, regeneration or thickening of lost or damaged hair extending to or beyond usual or expected anatomy.

“Hair restoration” refers to treatments including drugs, surgical techniques including but not limited to hair follicle transplantation (grafting), and other approaches that lead to an increase in the number, density, thickness, or general esthetic improvement of hair follicles or hair shafts. These favorable effects and responses are also referred to as hair restoration in the current document if they represent an attenuation of unfavorable changes in hair follicles or hair shafts, i.e., hair restoration may also reflect a slowing of hair loss.

“Therapeutic agents” as used herein refers to agents that are used to, for example, treat, inhibit, prevent, mitigate the effects of, reduce the severity of, reduce the likelihood of developing, slow the progression of and/or cure, a disease or other suboptimal or undesirable physical trait.

Insufficient bone formation relates to a situation in the body that results in a negative balance of bone homeostasis, in which the amount of bone lost or destroyed or catabolized is greater than the amount of bone the body makes. Bone formation may also be considered insufficient in cases where more bone is required to improve esthetics, as with facial bone augmentation, or to heal bone or stabilize an implant; examples include stable nonunions (no healing but no bone loss), no bone ingrowth (without bone loss), and no bone interdigitation of device to host bone (without bone loss). Nonunion is a state of persistent non-bridging of the fractured ends of a bone after the initial endochondral and/or intramembranous bone formation responses have ceased. Nonunions have a devastating impact on health-related quality of life, rivaling or exceeding that imposed by diabetes, stroke, or AIDs.

Bony fixation relates specifically to the adherence of an implanted device to the bone such that a stable construct exists with minimal motion or micromotion. It can relate to the fixation of a plate to a bone or a screw within a bone tunnel or an implant inside a medullary cavity or a prosthesis fit within a socket or a tissue, ligament or tendon surgically apposed or attached to bone or osseoincorporation (bone ingrowth and bone on-growth) of a porous or absorbable or composite device.

### Composition

A composition is provided including (1) an anti-DKK1 agent or another promoter of Wnt signaling and (2) an autologous body material (ABM) or non-autologous tissue such an allogeneic, xenogeneic, synthetic, or recombinantly-produced tissue that is functionally equivalent to or acts in a directionally similar manner, to a greater, or equal, or lesser degree, as an autologous version of the ABM.

Another composition is provided including an anti-DKK1 agent or another promoter of Wnt signaling and (2) an ABM or non-autologous tissue which has been treated or prepared to have at least 50%, 55%, 60%, 65%, 70%, 75%, 80%, 85%, 90%, or 95% of DKK1 or another Wnt antagonist removed. Exemplary Wnt antagonists include but are not limited to DKK1, sclerostin, secreted frizzled-related proteins (sFRPs), Wnt inhibitory factor 1 (WIF-1), and Wise.

In various embodiments, a promoter of Wnt signaling pathway is a protein, a peptide, a nucleic acid or a small molecule or a recombinantly-produced product. In some embodiments, the promoter of Wnt signaling pathway is an anti-DKK1 agent, such as a DKAB. In some embodiments, an anti-DKK1 agent is a molecule with complementarity-determining region (CDR) that binds directly to DKK1, such as but not limited to antibodies, peptibodies, camelbodies, nanobodies, peptides, phage-derived peptides, single domain antibodies, single-chain variable fragments (ScFv), antigen binding fragments (FAB), bifunctional antibodies or other related dimeric proteins or peptides wherein at least one of the antigen binding domains binds to or otherwise inhibits the activity of DKK1; CDR-containing molecules against DKK1-derived peptides, including peptides that mimic the LRP5/6 binding site within DKK1; gene therapy vectors, microRNAs, short hairpin RNAs, or oligonucleotides that interfere with the production or secretion of functional DKK1; competitive antagonists that interfere with DKK1-mediated inhibition of Wnt signaling; decoy molecules, receptors, or dominant-negative versions of molecules that interfere with DKK1-mediated inhibition of Wnt signaling; small molecules that interfere with DKK1-mediated inhibition of Wnt signaling; lithium chloride and other agents that over-ride DKK-mediated inhibition of Wnt signaling by acting downstream of Wnt receptors.

In other embodiments, a promoter of Wnt signaling is an agent that acts via molecular mechanisms other than via the inhibition of DKK1, which has the potential of overcoming the effects of DKK1 by activating or potentiating Wnt signaling via other molecule mediators. In some examples, a promoter of Wnt signaling pathway is lithium chloride (LiCl), which activates Wnt signaling by preventing β-catenin degradation within the cells. In other cases, recombinant Wnts can be used to activate Wnt signaling, including Wnt3a, which promotes the osteogenic differentiation of cultured bone marrow-derived mesenchymal stem cells (BM-MSCs). Local liposome-based administration of recombinant Wnt3a promoted bone healing in skeletal defects in mice.

Other embodiments provide that a promoter of Wnt signaling pathway is an agent that blocks other inhibitors of Wnt signaling besides DKK1, such as sclerostin, WIF-1, Wise/SOSTDC, or sFRPs. Such agents have the potential to overcome or overwhelm or otherwise compensate for the Wnt-pathway-inhibiting effects of DKK1. As with anti-DKK1 agents, agents that act by binding and inhibiting other soluble extracellular inhibitors of Wnt signaling have the potential to promote bone formation, hair growth, or wound healing without necessarily leaving the site of local delivery, or leaving it on a certain schedule, in contrast to agonistic drugs such as teriparatide, which have limited efficacy while still contained within delivery matrices.

An anti-DKK1 agent is provided to promote bone gain when administered locally to a subject. Inhibitors of DKK1, defined and described previously, include but are not limited to various therapeutic antibodies (DKAB) that bind to and inhibit the antagonistic action of DKK1, including various antibodies and—other proteins such as peptides, peptibodies, single-domain antibodies, bifunctional antibodies that bind to DKK1 with sufficient affinity and avidity as to prevent or reduce DKK1 antagonism of Wnt signaling. Inhibitors of DKK1 also include agents that act as a decoy or in a dominant-negative manner or as a competitive antagonist that limits DKK1-induced Wnt signaling inhibition without necessarily attaching to DKK1 itself, for example by occupying sites on Wnt receptors that limit DKK1 binding to said receptors. Inhibitors of DKK1 also include gene therapy vectors and other nucleotide-based agents that limit DKK1 synthesis, expression, production, or secretion.

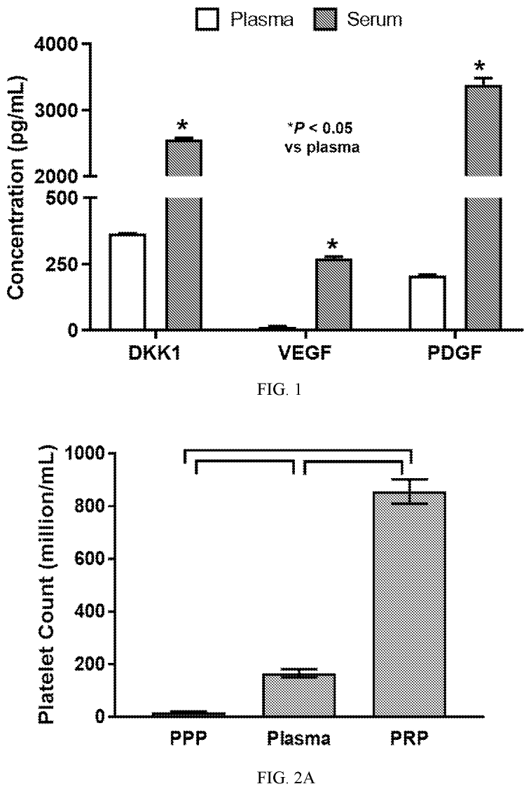

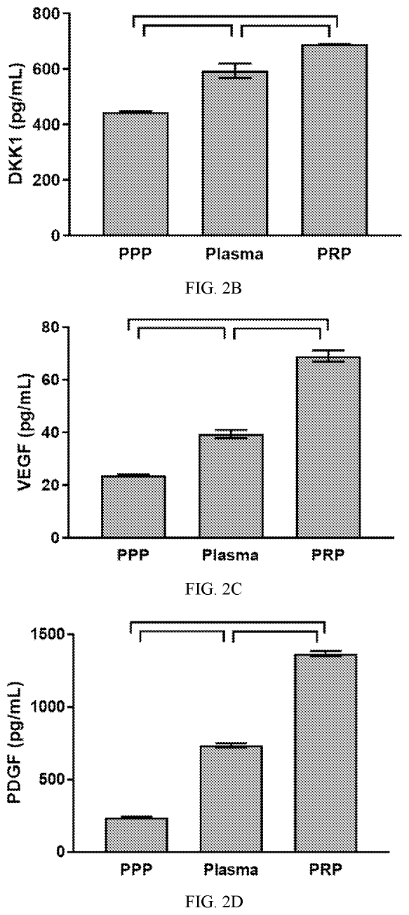

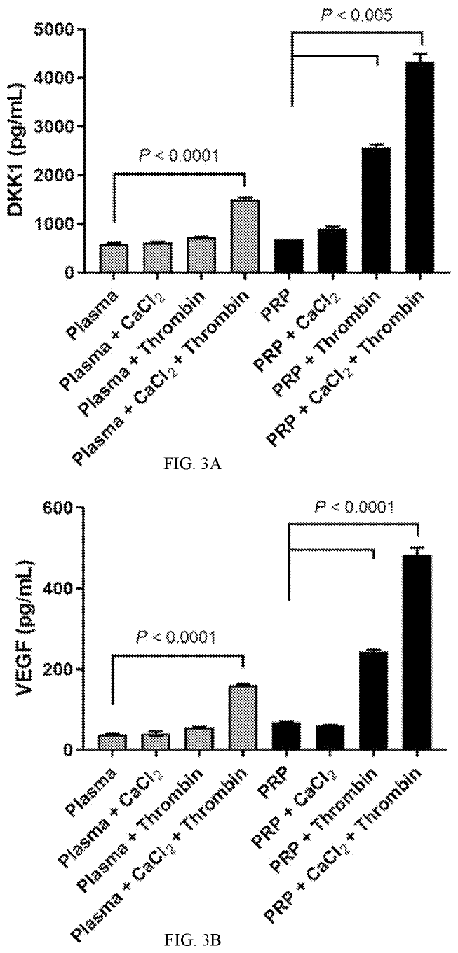

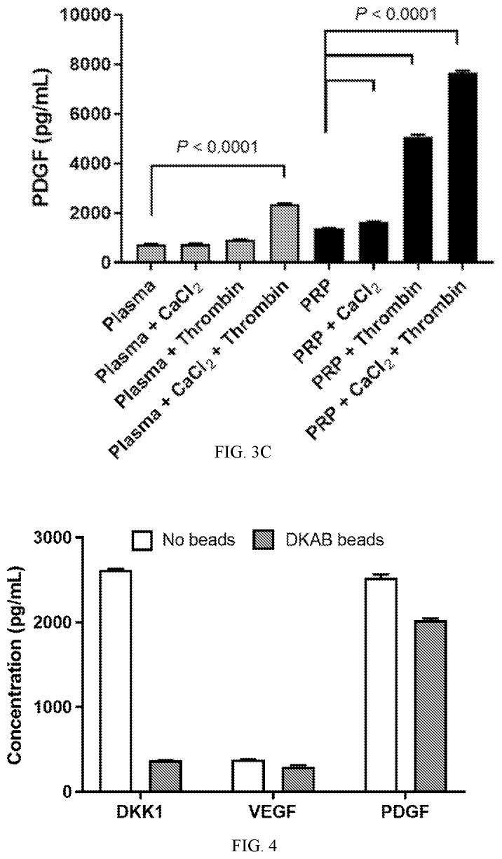

Exemplary autologous body materials (ABMs) suitable for inclusion in the composition, along with a promoter of Wnt signaling, include PRT, PRP, activated PRP, PRF, platelet gel, fibrin gel, activated plasma, PPP, recombinant or purified or autologous fibrin, bone marrow, BMA, BM-SVF, BMSCs, BMCs, BMACs, MSCs, BM-MSCs, AD-MSCs, MAT, AD-SVF, bone autograft, surgical bone, surgical blood, peripheral blood, local blood, MA, or any form of body material containing blood, serum, platelets or stroma; fresh or frozen. FIG. 1, FIGS. 2A-2D, and FIGS. 3A-3C show that serum, activated plasma, PRP, and activated PRP are all significantly enriched in the platelet-released growth factors VEGF and PDGF, as well as DKK1, compared with normal plasma. Elevated DKK1 levels in those ABMs indicate a suboptimal milieu for bone augmentation, wound healing, hair growth, and potentially other regenerative responses. FIG. 1 shows that concentrations of DKK1, VEGF, and PDGF are much higher in normal human serum compared with normal human plasma. This finding likely relates to platelet activation that inevitably occurs during serum preparation, whereas the anticoagulant used for plasma preparation inhibits platelet activation, such that internal stores of DKK1, VEGF, and PDGF remain largely within the platelets. DKK1 concentrations in serum (FIG. 1) were around 25-fold higher than the minimum DKK1 concentrations shown to inhibit bone formation in previous cell culture experiments (Li J, et al. Bone 2006; 39:754-766). These results suggest that blood coagulation (for example, in a fracture hematoma) induces sufficient DKK1 release from platelets to regulate (i.e. inhibit) certain healing responses. Indeed, several animal studies show that DKAB therapy promotes fracture healing, indicating that DKK1 inhibition improves the internal regenerative milieu, presumably by relieving DKK1-mediated blunting of the natural healing responses to endogenous growth factors and other regenerative factors. FIGS. 2A-2D demonstrate that DKK1 levels can become counterproductively high in certain ABMs that, unlike serum, do not involve natural platelet activation or clotting during their preparation. Higher platelet counts in plasma versus PPP, and in PRP versus plasma, were accompanied by stepwise increments in DKK1 levels, which was also the case with therapeutically beneficial platelet-released growth factors (e.g. VEGF and PDGF). The higher DKK1 and GF levels in these ‘unstimulated’ PRP and plasma samples versus PPP may reflect a low level of spontaneous or incidental DKK1 and GF release from their more numerous platelets. These results indicate for the first time that platelet enrichment carries with it an increase in the ABM's DKK1 levels, which may limit therapeutic benefits of concomitantly increased GFs levels in settings where greater Wnt signaling is desired, such as for bone augmentation, wound healing, and hair restoration. For ABMs that contain fibrinogen, both clotted (coagulated) and unclotted forms may be used, noting that some fibrinogen-containing ABMs may convert to fibrin gel-like forms when exposed to tissue upon their administration to patients. Many of these ABMs also have ‘functionally equivalent’ allogeneic, xenogeneic, recombinant, or synthetic versions with lesser, equal, or greater but directionally similar effects as an autologous version, that could also be combined with an anti-DKK1 agent or other promoter of Wnt signaling and administered locally to promote bone formation, bone augmentation, wound healing, or hair growth. These functionally equivalent materials include allogeneic forms of PRP, PRF, fibrin gel, BM-MSCs, AD-MSCs, whole blood, clotted blood, and allograft bone or stroma. Also included as functionally equivalent materials are recombinant or purified fibrinogen or fibrin in native or cross-linked forms, and various bone graft substitutes or bone void fillers, including allograft bone putty, demineralized bone matrix, synthetic fillers, silica, calcium-phosphate, hydroxyapatite, silk, collagen, or other absorbable or non-absorbable filler material, fresh frozen plasma, fresh frozen platelets, serum; any combination of fresh or frozen or otherwise mixed.

Some embodiments provide that ABM is platelet-rich plasma (PRP), which includes leukocyte-rich PRP (LR-PRP), leukocyte-poor PRP (LP-RPR), activated pure PRP (AP-PRP), activated PRP (A-PRP), and other forms for use of the present invention including in orthopedics, craniomaxillofacial surgery, dentistry, wound care, and hair restoration.

Some embodiments provide that ABM is platelet-rich fibrin (PRF), sometimes referred to as platelet gel, for use of the present invention including in orthopedics, craniomaxillofacial surgery, and dentistry. PRF can be made either by the centrifugation of whole blood collected without an anticoagulant, or by adding exogenous coagulation factors to plasma or PRP, which leads to platelet activation and the formation of a dense fibrin clot that is further enriched in soluble platelet-secreted factors. Platelet-released growth factors trapped in the fibrin matrix will be gradually released at the site of PRF administration by the fibrinolytic action of plasmin, leading to more sustained local growth factor exposure. Adding an anti-DKK1 agent or other promoter of Wnt signaling to the ABM prior to its activation may also lead to the entrapment of a proportion of said agent or other promoter within the resulting fibrin matrix, which may also be released via fibrinolysis. PRF's gel-like fibrin matrix can also be exploited to achieve controlled release of exogenous growth factors and other therapeutic agents, including BMP-2. PRF also has the utility of being able to bind bone graft materials together to enhance their local containment and retention. PRF generally has neutral-to-favorable effects on soft tissue healing and pain. PRF has been shown to improve the healing of bone grafts in alveolar bone, but more recent evidence suggests modest and inconsistent bone augmentation responses to PRF therapy. PRF had favorable effects on some aspects of tendon-bone healing in a goat ACL reconstruction model, but PRF did not promote tendon healing or improve clinical outcomes in randomized clinical studies of rotator cuff repair, and PRF had minimal effects on fracture healing. PRF did not improve implant fixation when mixed with a BMP2 gene therapy vector, nor did PRF increase the fusion rates of instrumented posterolateral spine fusions. We hypothesized and proved that the platelet activation step involved in preparing plasma gel (from plasma) or PRF (from PRP) leads to a dramatic increase in DKK1 levels, as it does for desirable platelet-released growth factors such as VEGF and PDGF (FIGS. 3A-3C). The higher levels of DKK1 in those ABMs, with or without elevated levels of endogenous DKK1 that often exists in bodily regions where PRF is eventually applied to promote bone augmentation, or wound healing, or hair restoration, would potentially limit the therapeutic effects of the ABM and the overall desired tissue responses. DKK1 concentrations in plasma and PRP activated with calcium chloride plus thrombin (FIGS. 3A-3C) were 15-fold and 40-fold higher (respectively) than the previously-reported minimum concentration of recombinant DKK1 that significantly impaired bone formation in vitro (Li J, et al. Bone 2006; 39:754-766). DKK1 concentration in these activated PRP samples was also similar to the elevated levels previously reported to be secreted by stromal cells harvested from non-union fractures, which were implicated in their impaired osteogenic differentiation in vitro. The degree of DKK1 induction in activated versus inactivated PRP was around 7-fold (FIGS. 2A-2D), and previous cell culture data using bone marrow samples from patients with multiple myeloma indicate that increments in DKK1 levels as little as 50% above normal are sufficient to impair osteogenic differentiation. Together, these findings indicate that both the absolute DKK1 levels and the relative degree of DKK1 enrichment in ABMs that undergo a platelet activation step are well within ranges that can impair Wnt signaling. This limitation could be addressed by adding an anti-DKK1 agent or another promoter of Wnt signaling pathway to the ABM. If it is desired to reduce DKK1 bioavailability within the PRF itself, without administering a DKK1 inhibitor to the patient, an immobilized DKK1-binding agent such as DKAB could be used to strip or deplete the ABM of much of its soluble DKK1, without the DKK1-binding agent being administered to the patient. FIG. 4 demonstrates that certain reagents such as DKAB-coated magnetic beads can be used to deplete most DKK1 from human serum while having minimal effects on levels of therapeutically-beneficial growth factors such as VEGF and PDGF.

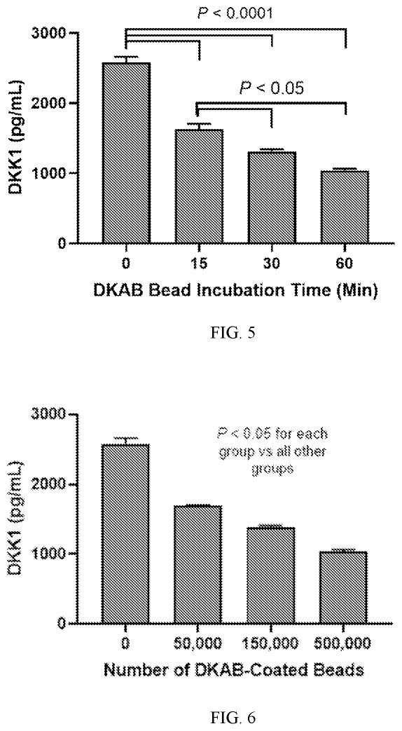

Some embodiments provide that ABM is fibrin gel, fibrin glue, or activated PPP, and the composition contains (1) fibrin gel, fibrin glue, or activated PPP and (2) a promoter of Wnt signaling. These ABMs are generally made by allowing or promoting fibrin cross-linking of platelet-poor plasma or recombinant or purified fibrin or fibrinogen. These materials have limited effects on bone formation, but their local application at bone injury and bone repair sites can provide a provisional matrix that supports angiogenesis and bone formation. These materials are gradually degraded in vivo by various enzymes, an attribute that fostered interest in their use for the controlled delivery of therapeutic agents, including PTH, BMP-2, and VEGF. There are currently no published reports on the use of fibrinogen-based gels or matrices to deliver an anti-DKK1 agent or other promoter of Wnt signaling, but such approaches could provide sustained local release of platelet-released growth factors as well as the anti-DKK1 agent or other promoter of Wnt signaling at sites of bone injury, or wound healing, or other settings, while also providing a provisional matrix to support cell-mediated tissue regeneration. Recombinant or purified fibrinogen, while not an autologous body material per se, can be activated to form cross-linked fibrin by agents such as thrombin, which is also commonly used to activate platelets and to create autologous fibrin gel. Thus, recombinant or purified fibrinogen can be used to create a fibrin gel that may be functionally equivalent to that which derives from activating autologous or allogeneic PPP, or plasma, or PRP. When treating injury or surgery sites where high endogenous DKK1 levels are expected, it may be particularly advantageous to incorporate an anti-DKK1 agent such as DKAB into a fibrin matrix or fibrin gel or clotted/clotting blood, or in some cases to ABMs with gel-like or cohesive properties derived from collagen-based reticular fiber networks, such as adipose tissue or bone marrow. This special advantage is based on evidence that steady-state binding of DKK1 to DKAB is not an instantaneous phenomenon: FIG. 5 shows that DKK1 levels were significantly reduced when human serum was incubated with DKAB-coated beads for 30 or 60 minutes compared with 15 minutes. These results suggest it may take an hour or more for DKK1-DKAB binding to reach a steady state, at least under those experimental conditions. Embedding an anti-DKK1 agent in fibrin matrix or gel would temporarily confine and retain the agent at its local site of administration, where DKK1 concentrations are highest and most problematic for local regenerative responses. This embedding feature may provide more time to achieve maximal DKK1 binding and neutralization. FIG. 6 shows that DKK1 depletion from human serum is more effective with increasing concentrations of DKAB-coated beads, indicating that the extent of DKK1 binding (and therefore inhibition) is also a function of DKAB concentration. Embedding an anti-DKK1 agent in a fibrin matrix or gel or other gel-like ABM would confine and retain the agent at the site of administration, thereby providing transiently higher local concentrations of the anti-DKK1 agent in the vicinity where DKK1 levels may be highest, thereby maximizing the potential for local DKK1 inhibition. The embedding of an anti-DKK1 agent in a fibrin matrix or gel or another ABM-associated matrix or gel would be particularly advantageous if the ABM itself is rich in DKK1, in that the anti-DKK1 agent and the ABM-associated DKK1 would remain in close physical association, providing greater opportunities for the DKK1 to become bound to and hence inactivated by the anti-DKK1 agent. The anti-DKK1 agent could be locally confined and retained by its addition to a fibrinogen-containing ABM that then undergoes conversion to fibrin matrix or fibrin gel through exposure to agents, tissues, or materials that activate platelets, or that induce fibrin formation and/or cross-linking. This conversion may be induced (ex vivo or in vivo) through the introduction of exogenous platelet activators including thrombin and/or calcium chloride, or may occur in vivo, without adding exogenous activators, as the result of exposure of the agent-ABM mixture to endogenous factors at the site of administration, such as thrombin, collagen, or tissue factor.

Some embodiments provide that the composition contains an autologous bone marrow (BM) and one or more agents that promote Wnt signaling, one of which may be an anti-DKK1 antibody. BM is a source of pluripotent mesenchymal stem cells (MSCs) that can be used in various forms for local bone augmentation. MSCs can differentiate into osteoblasts, chondrocytes, tenocytes, myocytes, or adipocytes, depending on the milieu to which they are exposed. BM is considered to provide much of the osteoinductive capacity of autologous bone grafts. Local MSC therapy was shown to have some favorable effects on hip osteonecrosis, on bone surrounding total joint replacements, and on alveolar ridge augmentation, whereas BM-derived MSCs did not improve tendon-to-bone healing in rotator cuff repair. BM can be an effective adjuvant for non-autogenous bone grafting, but several limitations make BM-based therapies suboptimal for bone augmentation. Firstly, the osteogenic capacity of bone marrow aspirates (BMA) tends to decline with advancing age. Furthermore, blood that contaminates BMA will contain platelets that may release DKK1 during the harvesting procedure itself, or after the BMA is re-injected to sites of tissue damage. The aqueous nature of BM creates challenges in containing and confining its benefits to defect sites, a limitation that may been addressed by allowing or inducing BMA clotting before it is applied. Clotted BMA also has the potential to control and sustain the release of drugs that are mixed into BMA before, during, or after clotting occurs. However, the clotting of BMA may also lead to the release of DKK1 from its platelets, which could be mitigated by the addition of an anti-DKK1 agent or overcome via the addition of another promoter of Wnt signaling. Another limitation is that only a small proportion of nucleated cells in adult BMA become osteogenic cells, which may explain in part why BMA generally has less osteogenic capacity compared with autologous iliac crest bone graft. Based on that limitation, centrifuges and blood separator machines can be used to make concentrated forms of BM, which may allow more MSCs to be delivered within the injury site. The process of BMAC preparation also leads to substantial (˜5-fold) enrichment in platelets, which may release growth factors and DKK1 that exert opposing influences on their proliferation and osteogenic differentiation.

The presence of elevated DKK1 levels occurring during bone surgery or from bone damage and at injury sites may explain why BM and BM cells have inconsistent and suboptimal effects on the healing of fractures and various bony defects. Bone anabolic agents have been added to bone marrow cells to increase osteogenesis, and the addition of an anti-DKK1 agent or other promoter of Wnt signaling pathway to BM, BMCs, BMA, or BMAC may also enhance osteogenesis by neutralizing DKK1 to favor the differentiation of BMCs along the osteogenic lineage. The addition of an anti-DKK1 agent or other promoter of Wnt signaling pathway to these cell-based ABMs would also create a more osteogenic local milieu after the anti-DKK1-ABM is applied to the patient, thereby promoting osteogenesis by the resident population of BMCs, osteoprogenitors, and osteoblasts. As is the case with stem cells in general, BM-derived MSCs are minimally immunogenic, and thus allogeneic BM-MSCs may be considered a functional equivalent of autologous BM-MSCs, either of which could be combined with an anti-DKK1 agent or other promoter of Wnt signaling pathway based on the rationales described above.

Some embodiments provide that the composition contains adipose tissue and one or more agents that promote Wnt signaling one of which may be an anti-DKK1 agent. Adipose tissue is another source of MSCs and can be harvested via liposuction or other techniques. The micronized adipose tissue (MAT) or adipose-derived stromal vascular fraction (AD-SVF) is capable of osteogenic differentiation. Adipose-derived MSCs have been used for various clinical conditions, based in part on the higher yield of such cells from adipose tissue compared with BMA. MAT or AD-SVF, which are enriched in MSCs, improved bone regeneration in segmental defects. Human adipose tissue and preadipocytes were both shown to express DKK1, and the adipogenic differentiation of preadipocytes was associated with increased DKK1 expression and reduced Wnt signaling. Forced over-expression of DKK1 promoted the adipogenic differentiation of preadipocytes, whereas DKK1 inhibition promoted the osteogenic differentiation of adipose-derived MSCs. As such, the addition of an anti-DKK1 agent or other promoter of Wnt signaling to adipose tissue, AD-MSCs, or MAT or AD-SVF could promote osteogenic differentiation of the MSCs contained within those sources, while also delivering the anti-DKK1 agent or other promoter of Wnt signaling to the injured tissue to promote osteogenesis from resident cells. As is the case with stem cells in general, AD-derived MSCs are minimally immunogenic, and thus allogeneic AD-MSCs may be considered a functionally equivalent version of autologous AD-MSCs, either of which could be combined with an anti-DKK1 agent or other promoter of Wnt signaling based on the rationales described above.

Some embodiments provide the composition contains autologous whole blood, an agent that promotes Wnt signaling, and further optionally an anti-DKK1 antibody. Autologous whole blood is for use in procedures where osteogenesis is desired. The re-administration of autologous peripheral venous blood under the maxillary sinus membrane at the time of dental implant placement can increase bone formation in the maxilla. The basis of this effect may involve the release of various factors from activated platelets that stimulate angiogenesis and osteogenesis, and/or the provisional matrix provided by the blood clot that forms after administering whole blood to sites of injury. Whole blood can be added to graft material to provide better cohesion of graft particles, thereby improving their physical handling characteristics in the hands of surgeons and other clinicians, while also limiting the risk of dispersal and migration of graft particles after their placement within the patient. Surgical blood salvaged intra-operatively is a convenient alternative to whole venous blood, as no venipuncture is required; the recovery and re-use of surgical blood may also limit the need for blood transfusions. Salvaged surgical blood, which appears similar to peripheral blood in terms of platelet concentration and growth factor levels, has been used to make PRP for spinal fusion surgery. Whereas peripheral venous blood promoted alveolar bone augmentation, clotted blood had no significant osteogenic effect when delivered to extraction sockets. Clotted blood at sites of bone injury was suggested to impair osteogenic responses to endogenous BMPs via inhibitory effects of platelet-derived PDGF or FGF. But considering that PDGF and FGF each have potent bone anabolic effects on their own, whereas DKK1 is a potent inhibitor of osteogenesis, it is possible that platelet-derived DKK1 plays a key role in limiting the osteogenic efficacy of clotted blood. However, the ability of an anti-DKK1 agent or other promoter of Wnt signaling pathway including DKAB to ameliorate inhibitory effects of clotted blood on osteogenesis has never previously been tested or demonstrated. Systemically-administered DKAB can promote alveolar bone regeneration after tooth extractions, which is consistent with the possibility that DKK1 secreted from activated platelets within whole blood, particularly clotted blood, and from resident platelets within damaged bone, may limit local osteogenesis. The addition of an anti-DKK1 agent or other promoter of Wnt signaling pathway to autologous whole blood products may enhance local osteogenesis compared with whole blood itself via the effects of the anti-DKK1 agent or other promoter of Wnt signaling pathway on DKK1 found within the blood product itself, and/or by improving the local osteogenic milieu where those blood products are applied.

Some embodiments provide the composition contains autologous bone and one or more agents that promote Wnt signaling, one of which may be an anti-DKK1 agent. Autologous bone is considered the gold standard bone grafting material. Autologous bone is osteoinductive due to its BMP-2 and other growth factors, is osteogenic due to its osteoprogenitor cells, and also serves as an osteoconductive scaffold that supports angiogenesis and osteogenesis. Bone autografts are often harvested from the iliac crest, but also from the vertebrae, ribs, tibia, fibula, chin, mandibular ramus, and other sites. Bone autografts in the form of blocks, strips, morsels, chips, and other forms are extensively used in orthopedics, spine surgery, craniomaxillofacial surgery, and dentistry. Major limitations of bone autografts include the need for an additional surgical site and the resulting donor site morbidity. Furthermore, the amount of available autograft material can be limiting, and autografts from older individuals may have reduced osteogenic capacity. Attempts to improve the efficacy of bone autografts include the co-delivery of bone anabolic agents, either systemically or locally. Numerous alternatives to autologous bone grafts have also been developed, including various allografts and bone graft substitutes such as demineralized bone matrix and calcium-phosphate products, including hydroxyapatite. Graft substitutes are often supplemented with growth factors or MSCs to overcome their limitations as non-vital materials. While no such attempts have yet been described, the addition of an anti-DKK1 agent or other promoter of Wnt signaling pathway to bone grafts or bone graft substitutes may enhance their osteogenic properties. Bone autograft can be harvested opportunistically during certain surgical procedures, thereby eliminating the need for an additional surgical site and associated morbidity. For example, intra-operative bone autograft can be obtained from vertebrae during certain spinal fusion procedures. Bone autograft material in the form of bone fragments and marrow can also be harvested in the form of reamer-irrigator aspirate (RIA) when the medullary canal of a long bone is reamed to accommodate intramedullary nailing and fixation. Femoral reaming via intramedullary nail caused a substantial increase in growth factors within the femoral canal, including VEGF, PDGF, IGF-I and TGF-β, and these same growth factors are enriched in PRP. These findings are consistent with the possibility that platelet activation during reaming may cause the release from platelets of anti-osteogenic DKK1. The addition of an anti-DKK1 agent or other promoter of Wnt signaling pathway to bone autograft materials, bone graft substitutes, or RIA has the potential to augment their osteogenic capabilities, whether by acting on graft-associated cells that possess osteogenic activity or potential, and/or by acting on resident cells in the injury site or upon the target milieu after placement of the graft, graft substitute, or RIA. A variety of bone graft substitutes exist, including allogeneic freeze-dried or irradiated human bone, bovine collagen, collagen sponges, calcium-phosphate granules, calcium phosphate putties, hydroxyapatite granules, bone void fillers, etc. Many of these materials can be functionally equivalent to autologous bone grafts, especially when delivered in conjunction with pro-osteogenic factors or growth factors such as BMP-2, which provides a rationale for combining these materials with an anti-DKK1 agent or other promoter of Wnt signaling pathway to achieve bone augmentation and graft incorporation.

An anti-DKK1 agent or other promoter of Wnt signaling and an ABM or a functionally equivalent non-autologous material can be combined in one or more of the following ways: mixing, stirring, agitating, centrifuging, agitating, injecting, mechanical transfer, pouring, spraying, painting, coupling, and direct co-application; or other form of cell separation or mixing technique such as electrophoresis, or electroplating, or conductive or other non-mechanical means.

Typically, an anti-DKK1 agent or other promoter of Wnt signaling is combined with an ABM or non-autologous functional equivalent at a concentration of at least 0.1-0.5 wt/wt %, 0.5-1 wt/wt %, 1 wt/wt %, 2 wt/wt %, 3 wt/wt %, 4 wt/wt %, 5 wt/wt %, 6 wt/wt %, 7 wt/wt %, 8 wt/wt %, 9 wt/wt %, 10 wt/wt %, 10-15 wt/wt %, 15-20 wt/wt %, 20-30 wt/wt %, 30-40 wt/wt %, 40-50 wt/wt %, 50-60 wt/wt %, 60-70 wt/wt %, 70-80 wt/wt %, 80-90 wt/wt %. In some embodiments, a promoter of Wnt signaling pathway (e.g., an anti-DKK1 agent) is combined with an ABM (or an allogeneic material) at a concentration of at least 0.1-0.5 vol/vol %, 0.5-1 vol/vol %, 1 vol/vol %, 2 vol/vol %, 3 vol/vol %, 4 vol/vol %, 5 vol/vol %, 6 vol/vol %, 7 vol/vol %, 8 vol/vol %, 9 vol/vol %, 10 vol/vol %, 10-15 vol/vol %, 15-20 vol/vol %, 20-30 vol/vol %, 30-40 vol/vol %, 40-50 vol/vol %, 50-60 vol/vol %, 60-70 vol/vol %, 70-80 vol/vol %, 80-90 vol/vol %. In other embodiments, an anti-DKK1 agent or other promoter of Wnt signaling is combined with an ABM or non-autologous functional equivalent at a concentration of at least 0.1 mg/mL, 0.1-1 mg/mL, 1-5 mg/mL, 5-10 mg/mL, 10-20 mg/mL, 20-30 mg/mL, 30-40 mg/mL, 40-50 mg/mL, 50-60 mg/mL, 60-70 mg/mL, 70-80 mg/mL, 80-90 mg/mL, or 90-100 mg/mL or greater than 100 mg/mL.

In some embodiments, the composition includes more than one agent capable of promoting and/or potentiating Wnt signaling, such as an anti-DKK1 agent combined with a recombinant Wnt (e.g. liposomal Wnt3a); or an anti-sclerostin antibody combined with an R-spondin that potentiates Wnt signaling; or an anti-DKK1 agent combined with an anti-SFRP antibody; or Wnt3a combined with an R-Spondin or an anti-Wise/SOSTDC antibody. These agents can be added to autologous body materials such as PRP, or directly injected into ABMs in situ, for example a fracture hematoma that is rich in activated platelets that are expected to transiently increase local DKK1 levels. The combination of more than one promoter of Wnt signaling is expected to overcome the inhibitory effects of platelet- and osteocyte-derived DKK1, leading in some cases to greater Wnt signaling than would be achieved by the inhibition of DKK1 alone.

### Pharmaceutical Composition/Formulation

In various embodiments, the present invention provides a pharmaceutical composition. The pharmaceutical composition includes or consists of (1) an anti-DKK1 agent, e.g., an anti-DKK1 antibody and/or other promoters of Wnt signaling, (2) an ABM or functional equivalent, which optionally has been treated or prepared to have at least 50%, 55%, 60%, 65%, 70%, 75%, 80%, 85%, 90%, or 95% of DKK1 or another Wnt antagonist removed, and (3) a pharmaceutically acceptable excipient. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous. Examples of excipients include but are not limited to starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof.

In various embodiments, the pharmaceutical compositions may be formulated for delivery via any route of administration. “Route of administration” may refer to any administration pathway known in the art, including but not limited to aerosol, nasal, oral, transmucosal, transdermal, parenteral or enteral. “Parenteral” refers to a route of administration that is generally associated with injection, including but not limited to percutaneous, subcutaneous, intravenous, infusion, transdermal, intraarterial, intraarticular, intracapsular, intracardiac, intraosseous, intradermal, intramuscular, intraperitoneal, intrapulmonary, intraspinal, intrasternal, intrathecal, intrauterine, subarachnoid, subcapsular, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Typically, the compositions are administered by injection. Methods for these administrations are known to one skilled in the art.

The pharmaceutical compositions can contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid, gel, or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly per oral or filled into a soft gelatin capsule. A promoter of Wnt signaling (e.g., an anti-DKK1 agent) can be in one or more of the following forms: lyophilized, crystallized, liquid, frozen, gel, powder, paste, and emulsion.

In some embodiments, the pharmaceutical composition is administered locally. In some embodiments, the pharmaceutical composition includes a controlled release carrier for slow, attenuated, responsive or on-demand release of the promoter of the Wnt signaling pathway. Exemplary controlled release carriers include liposomes, polymeric particles, and natural or synthetic polymeric matrices. Methods of preparing liposome delivery systems are discussed in Gabizon et al., Cancer Research (1982) 42:4734; Cafiso, Biochem Biophys Acta (1981) 649:129; and Szoka, Ann Rev Biophys Eng (1980) 9:467. Other drug delivery systems are known in the art and are described in, e.g., Poznansky et al., Drug Delivery Systems (R. L. Juliano, ed., Oxford, N.Y. 1980), pp. 253-315; M. L. Poznansky, Pharm Revs (1984) 36:277. Exemplary polymeric micro- or nano-particles are prepared with poly lactic-co-glycolic acid (PLGA), poly lactic acid (PLA), poly glycolic acid (PGA), polyealkylene glycol, hyaluronic acid, collagen, and chitosan. After the liquid pharmaceutical composition is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is administered to subjects using those methods that are known to those skilled in the art.

### Methods of Preparation and Using

Exemplary techniques of applying a composition containing an anti-DKK1 agent and/or other promoter(s) of Wnt signaling and an ABM to a subject or to an implantable device include, but are not limited to, spraying, painting, dripping, aerosolizing, injecting (by any topical or parenteral route, including; subcutaneous, percutaneous, intravenous, intramedullary, intraosseous, or intradermal), directly applying to the body (via a cream, gel, glue, membrane, onlay, inlay, wrapping, filling, plugging, suturing, covering, containing, or barrier creation), dipping, immersing, coating, and soaking, or any other non-mechanical method to apply a material that may be based on electrical currents or magnetic properties or other conductive or electrophoretic method.

In some embodiments, the composition containing an anti-DKK1 agent and/or other promoter(s) of Wnt signaling and an ABM or functional equivalent are applied to an implantable device before, during, or after the device's implantation. Exemplary devices for inclusion of the composition include bone autografts, bone allografts, demineralized bone matrix, calcium- and or phosphonate-containing graft substitutes, space fillers, void fillers, hydroxyapatite-containing devices, strips, blocks, sponges, plugs, tapes, adhesives and various implantable device that undergo some degree of bony fixation such as; dental implants, orthopedic implants, plastic surgery implants, craniofacial implants, prosthesis, fusion cages, screws, screws, plates, pins, buttons, discs, wires, rods, either directly on the raw surface, either solid or porous, or over any modified surface or textured surface or over any coating applied to the surface of such a device. The anti-DKK1 agent and/or other promoter of Wnt signaling, the ABM, and the device maybe assembled either outside the surgical field via various standard manufacturing methods, within the sterile field by a technician according to the products instructions for use or in-vivo by the clinician, depending on the varying regulatory requirements or the application of the product or the desired effect or clinician discretion.

In other embodiments the product is applied to patients using a device that is identical, similar, or related to dual syringes that are commonly used to deliver activated PRP or activated plasma. Such devices have the utility of introducing agents that activate platelets or promote fibrin formation (e.g. thrombin and/or calcium chloride) to fibrinogen-containing ABMs such as plasma or PRP at a fixed stoichiometry (often ten parts ABM to one part activator) in a controlled and consistent manner. Force applied to the plunger of these dual syringes causes the co-expression and synchronous mixing of the two components, leading to consistent and homogeneous fibrin formation throughout the ABM. Various delivery tips or attachments used in conjunction with such dual syringe devices allow the resulting mixture, which can in some cases begin to congeal within seconds to minutes, to be passed from the device to the patient or an implant or graft as a spray, a stream, an aerosol, or a drip, or as a percutaneous injection, or as an arthroscopic, or minimally-invasive, or other route of injection. One alternative to this arrangement would place the ABM by itself in one barrel, and an admixture of aqueous DKAB solution plus activator (e.g. thrombin and/or calcium chloride) in the other barrel. These devices and their many potential applications have not previously been used to deliver therapeutic molecules such as anti-DKK1 agents or other promoters of Wnt signaling that are mixed with autologous fibrinogen-containing ABMs. It is also feasible to develop and utilize a triple-barrel syringe, with the ABM alone placed in one barrel, an aqueous solution containing an anti-DKK1 agent and/or other promoter of Wnt signaling in a second barrel, and an activating solution (e.g. thrombin and/or calcium chloride) in a third barrel, all at pharmaceutically suitable relative volumes.

In other embodiments, the anti-DKK1 agent and/or other promoter(s) of Wnt signaling combined with an ABM is applied topically or administered as an injection under or into the skin or into the scalp to promote hair growth, for example in patients with AGA, or to reduce hair loss in patients undergoing radiation or cancer-related treatments, or applied with a dermal covering, or fascia or other tissue extender to heal wounds or provide soft tissue coverings where clinically indicated. Microneedling, a hair restoration strategy that provokes bleeding, could also be followed by topical application of an anti-DKK1 agent or other promoter of Wnt signaling in combination with PRP or other ABMs. Hair grafts (plugs) could be immersed, soaked, or otherwise exposed to an ABM such as PRP that contains an anti-DKK1 agent or other promoter of Wnt signaling, or to an ABM from which DKK1 has been largely stripped (removed) via an immobilized DKK1-binding factor, prior to their placement in the scalp, to enhance the survival and vitality of transplanted hair grafts.

Various embodiments provide that an anti-DKK1 agent and/or other promoter(s) of Wnt signaling can be combined with an ABMs at various stages, e.g., at manufacturing, assembly, clinical or surgical stages. In some aspects, an anti-DKK1 agent or other promoter of Wnt signaling is pre-loaded into an ABM tissue preparation container, collector, or receptacle, such as PRP collection and preparation kit, an RIA collection apparatus, a bone or tissue harvesting kit or after the ABM has been collected, including at various steps of its ex vivo preparation, or after the ABM is fully prepared and otherwise ready for administration to the patient.

Further embodiments provide that a kit or a packaged system contains an anti-DKK1 agent or another promoter of Wnt signaling, and needles, reagents, vessels, transfers, disposables or other materials involved in obtaining and preparing the ABM of choice, and mixing, combining or otherwise applying or introducing the aforementioned agent(s) to the ABM. In one aspect, the kit has components that are sterile and for one-time use, and/or a closed system, and optionally further includes a delivery device designed to administer the agent and the ABM in a clinically appropriate manner. The delivery device includes, but is not limited to, a needle, a syringe, an injector or a spray device. Having an all-in-one system would support existing clinician and follow hospital practices for ease of use. It would reduce the time to find the correct equipment, prepare the surgical setting and prepare the composition. A closed, sterile, single-use system reduces the risk of product contamination from airborne substances and minimizes the risk of patient infection. An all-inclusive kit would also improve the quality control of the composition being delivered to the patient by establishing and providing a standard, reliable, reproducible and validated protocol, for producing said composition.

The composition containing an anti-DKK1 agent and/or another promoter(s) of Wnt signaling and an ABM is typically administered to a target tissue of interest in a subject. Exemplary targeted tissues include bone, or bone stroma or osseous tissue from long bones, facial bones, spine, pelvis, or cancellous bone or cortical bone or intramedullary bone of any kind in the body, or alveolar/mandible or facial bone. Other tissues include hair and skin and any bone or joint or muscle or tissue that requires a tissue covering or tissue extension such as in the case of ulcer treatment and joint capsular or tendon or ligament reconstruction.

Currently, one of the most challenging environments to create new bone is in nonunions. The costs and health economic consequences of nonunion treatment are very high, and the morbidity and reduced quality of life has significant impact to the patient, and to society. Healing a nonunion to its prior normalized functional state greatly improves the quality of life and accelerates or increases the likelihood of returning to work, sport, or activities of daily living. Accelerated healing also allows earlier weight-bearing and the initiation of physical therapy and exercise. In nonunions, there can be physiological and structural barriers that limit the fracture's healing while also limiting the efficacy of systemically-administered therapies. This is because the vascularity of the local bone inside the nonunion site is often disrupted or otherwise deficient, which is likely to limit the site's exposure to systemically-administered therapies. This unfavorable feature suggests significant advantages to the local over the systemic administration of bone-anabolic agents, and anti-DKK1 agents may be particularly beneficial for nonunions because they promote not only osteogenesis, but also chondrogenesis, a key event that creates the initial union of most long bone fractures. Furthermore, PRTs and certain other ABMs are highly enriched in VEGF (FIGS. 2A-2D and 3A-3C), indicating that PRT-based delivery of an anti-DKK1 agent or other promoter of Wnt signaling pathway into nonunions may have a synergistic effect, with the ABM-derived VEGF promoting early neovascularization that fosters delivery of the pro-osteogenic agent to previously avascular and osteogenically quiescent sites. Surgical interventions for nonunions typically include decortication and other preparation to achieve bleeding bone ends, which are likely to promote substantial platelet activation that increases local levels of growth factors and DKK1. The adverse effects of this platelet-released DKK1 could be directly inhibited or otherwise overcome by administering an anti-DKK1 agent and/or other promoter(s) of Wnt signaling into the site of surgical intervention, which itself is a (non-harvested) ABM by virtue of its growth factor-rich milieu. It may be preferable and more efficacious to administer the anti-DKK1 agent and/or other promoter(s) of Wnt signaling (with or without a previously harvested ABM) directly into the region of a freshly decorticated and prepared nonunion, as opposed to systemic administration at a remote site, because the latter routes will result in delayed or suboptimal local exposure to the agent during the key post-surgical period of transiently-elevated DKK1. It is also noteworthy that the form factors of PRP and most other ABMs described herein are amenable to delivery by percutaneous, arthroscopic, or minimally-invasive approaches, which is not feasible with several current bone-augmenting therapies such as recombinant PDGF-BB or BMP-2, which must be delivered with more rigid carriers at open surgical sites. This feature creates new possibilities of interventions in which anti-DKK1 agents or other promoters of Wnt signaling may be administered with ABMs to nonunion sites in association with limited decortication via small-incision or limited-exposure approaches. The feasibility of these less invasive procedures is predicated in part on the notion that the combined effects of decortication, growth factor release, and DKK1 inhibition are sufficient to generate robust healing responses that are currently achieved by more invasive, morbid, time-consuming and costly surgical procedures that also require longer recovery times.

When fractures heal, there is a phase-guided rehabilitation process to return the patient to a functional level. If the patient re-ambulates too soon prior to adequate local bone healing and consolidation, the fracture may cause substantial discomfort and may also re-break at the same site. A more conservative or apprehensive patient may be inclined to avoid such risks by taking a more cautious rehabilitation approach, which can lead to further delays in return to work, play, or daily activities, while also experiencing additional muscle atrophy and other deleterious health effects such as thromboses. A therapy that promotes or augments the local healing response in such a manner as to heal the bone more rapidly, with an accelerated regain of biomechanical function, will allow a patient to accelerate their rehabilitation with less discomfort, less apprehension, and a reduced risk of breaking the same bone again. In traditional care, without local treatment, a patient must wait a sometimes-prolonged period of time until there is radiographic evidence of bone bridging across the fracture site with clinical signs of healing such that the patient may safely begin bearing weight across the bone and progressing to the next phases of rehabilitation. As such, there exists an unmet need to promote and accelerate the healing of fracture even if they have minimal risk of progressing to delayed or nonunion. Most fractures are of a closed (non-compound) nature, and the ability to deliver anti-DKK1 agents or other promoters of Wnt signaling into a fracture site percutaneously or by other minimally invasive approaches, with or without an added ABM, is a feasible approach to accelerating the biomechanical recovery of routine closed fractures that might otherwise involve more prolonged or less predictable healing than the patient would desire.

In the case of bone infections at the site of implantation or fracture, there are often many factors resulting in the compromised state of the patient and the local site of tissue involved. In some cases, implant instability is identified to be a cause, and in other cases it can be attributed to excessive motion across the fracture site, lack of bone ingrowth around implant, or aseptic loosening. In all cases, promoting bone growth across the fracture site and increasing the amount of bone-implant-contact (BIC) may be critical. Implant osseointegration is also important. If a product existed that would improve the implant stability, fixation and adhesion, it is likely that those sources of failure would be averted. In such cases, early implant stability, accelerated fracture healing to reduce motion across the fracture site, decreased BIC and increased bone ingrowth and osseoincorporation would decrease the risk of treatment failure, implant failure, implant retrieval, implant loosening, infections, and numerous other complications. Optimal BIC and reduced micromotion by using an ABM an anti-DKK1 agent or other promoter of Wnt signaling pathway at the time of implant surgery may reduce such complications.

Another potential application of anti-DKK1 agents or other promoters of Wnt signaling involves improved implant osseointegration, the process by which implants such as spinal fusion cages or hip prostheses or dental implants become rigidly and durably connected to bone through deposition of new bone matrix into, onto, and/or around the implant. DKK1 inhibition via DKAB administration to rats was shown to promote the osseointegration of titanium screws, an effect that involved a clear interaction effect between DKAB administration and the bone injury that resulted from screw placement. This interaction indicates the existence of an acute biological response to injury that conferred therapeutic responsiveness to DKAB. This response could potentially involve DKK1 release from activated platelets and/or perturbed osteocytes at the site of screw placement. Regardless, local delivery of an anti-DKK1 agent or other promoter of Wnt signaling at the site of implant placement has the potential to immediately create high drug levels to better inhibit or overcome the deleterious effects of transiently elevated DKK1 levels. Most approaches to achieve faster and more robust implant osseointegration involve engineering-based modifications to implant surfaces or topographies to encourage bony ingrowth and on-growth, including metal surface technologies such as grit-blasting, plasma-spray coating, sintering beads and fiber-metal. Recent advances have focused on creating three-dimensional materials that include trabecular metal, titanium foam and various 3D printed titanium, PEEK and ceramic materials. While these advances in technologies and materials have produced improved scaffolds for bone to grow into, they have done little to accelerate the rate nor density of bone generation. Biological approaches to promoting implant osseointegration are varied, with minimal successes. Numerous investigations tested the ability of PRTs including PRP to promote implant osseointegration, with inconsistent results. One notable study showed that the osseointegration of titanium screws place in the femur of rabbits was moderately improved by the application of PRP with moderate platelet enrichment, whereas PRP with the greatest degree of platelet enrichment led to impaired osseointegration. The investigators speculated that this “astonishing” paradoxical effect may have been due to inhibitory or toxic effects of more highly concentrated growth factors, but it may be more plausible that hyper-enriched DKK1 contributed to the adverse effect. In support of this notion, DKK1 silencing was shown to enhance the osteogenic differentiation of cells placed on various titanium and modified titanium surfaces. Collectively, these findings point to a novel approach of promoting implant osseointegration by combining an anti-DKK1 agent or other promoter of Wnt signaling to PRTs, including PRP, and delivering them within or around or beneath or atop implants intended to undergo osseointegration. By this and related approaches, the peri-implant bone will be exposed to numerous endogenous growth factors in concentrated form, and the therapeutic agent with which the PRT was combined would inhibit, neutralize, or otherwise overcome the deleterious effects of DKK1 present within the PRT itself, and that which will be induced by the placement of the implant, whether from activated platelets or perturbed osteocytes. This local delivery approach has the added advantage of immediately placing the anti-DKK1 agent or other promoter of Wnt signaling at the site of local DKK1 induction, which is likely to be a transient injury-related phenomenon that may be less effectively inhibited had the same dose of the agent been administered systemically.

In the case of ligament injuries where the repair or reconstruction relies upon robust bone-to-bone healing or tendon-to-bone healing, new bone formation is required to interdigitate the surfaces to improve the extent and durability of fixation. Athletics is a high demand activity, particularly professional sports, where there is a high likelihood of injury which in many cases is career-ending or career-altering. There can be significant benefit and value in returning a competitive athlete to her or his pre-injury level of function in an accelerated time frame. Application or injection of an ABM and an anti-DKK1 agent or other promoter of Wnt signaling pathway after the injury or potentially prophylactically in some cases, may reduce risk of injury, improve healing, outcome and return to work or sport.

In some orthopaedic indications for bone disorders, a fusion is necessary across a joint that has failed, been destroyed, traumatized, infected, or been exposed to numerous other causes that may cause a symptomatic or dysfunctional debilitating bone joint disorder. In spine fusion, degenerative changes in the axial spine can lead to pain and destruction of joints that may eventually fail non-surgical care and require fusion. Achieving fusion in the spine can lead to improved clinical outcomes, but lack of fixation at anchor points above and below the joint, the need for limited activity during healing, and the potential loosening of hardware or pullout of screws represent significant therapeutic limitations. Fusion is typically augmented with bone autograft, the harvesting of which involves significant bleeding and blood loss at the surgical site, pain, and the risk of other significant complications. The local environment and the overall morbidity of the patient increases risk of implant failure. The longer it takes for a fusion to occur, the greater the risk of failure. Other joints that may be fused include the ankle, the sacroiliac, the wrist, carpo-metacarpal joints, elbow, shoulder, hip, and knee. Creating an optimal healing milieu at the fusion site is important, and the necessary surgical trauma involved in many arthrodesis procedures, including decortication to induce local bleeding and to increase exposure to bone marrow elements, may create a favorable yet suboptimal milieu if sufficient anti-osteogenic DKK1 is released from platelets and/or perturbed osteocytes. BMPs are often used as an adjuvant to spinal fusion, and factors released from activated platelets have been shown to significantly diminish BMP induced osteogenic differentiation. The identify of said factor(s) remains unknown, and according to this invention, DKK1 may be among the factors released by activated platelets that may contribute to diminished BMP-induced osteogenesis. An ABM plus an anti-DKK1 agent and/or other promoter(s) of Wnt signaling pathway may address problems with fusion.

Osteonecrosis may occur for a variety of reasons. With osteonecrosis, the loss of bone or subchondral bone and support for cartilage and joint surfaces is very problematic. There is no good surgical treatment for osteonecrosis apart from joint replacement, and while some systemic therapeutics can delay the need for arthroplasty, they do not foster the replacement of dead bone with living bone. In the case of osteonecrosis of the jaw (ONJ), there is no good treatment for the significant bone loss that can occur. After osteonecrotic bone anywhere in the skeleton is removed by osteoclasts and macrophages, the residual skeletal architecture comprising vital bone is frequently insufficient for normal load bearing or for the accommodation of certain surgical repair procedures. PRP has been studied for the treatment of osteonecrosis of the femoral head, the jaw, and other sites, often in conjunction with stem cell therapy, with mixed results. Anti-DKK1 agents and/or other promoter(s) of Wnt signaling combined with ABMs may be a suitable way to initiate or promote restoration of local bone structure and architecture via the formation of new bone.

Similarly, various other bone disorders have unmet needs related to poor bone formation or bone loss or structural insufficiency, including but not limited to bone cysts and oncology-related bone defects. Myeloma-related bone disease involves systemic bone loss and focal lytic bone lesions, and the ability of bone augmentation via DKK1 inhibition to address these skeletal deficiencies has been investigated through systemic DKAB administration. Alternatively, an anti-DKK1 agent such as DKAB could be administered locally to problematic lytic lesions in patients with myeloma, including lesions that may be causing or at risk of causing spinal cord compression or pathological fractures. In some cases, the anti-DKK1 agent may be mixed with an autologous plasma gel and/or bone graft particles and delivered during open or minimally-invasive surgery. Alternatively, the agent could be delivered as a local percutaneous, minimally-invasive, or intra-osseous injection as a way of avoiding more invasive and morbid surgical procedures. These local delivery approaches have the advantage of delivering a high amount of the anti-DKK1 agent to a lesion that is driven by aberrant DKK1 secreted by local myeloma cells, which would likely provide greater target coverage (i.e. DKK1 inhibition) compared with delivering the same dose of the agent systemically, such as by subcutaneous or intravenous injection.

Craniofacial, dental and maxillofacial problems may also be treated with the composition described herein for bone formation or reconstitution. Alveolar bone deficiency and implant site development is also suitable for treatment with the composition described herein.

In the case of wound healing, there is a vast need to treat difficult and challenging wounds such as diabetic ulcers, chronic draining sinuses, infected wound beds, burns, exposed bone or tendon ulcers or wound dehiscence in patients with medical comorbidities. While there are numerous dressings and topical treatments currently on the market to address chronic wounds, the field of wound care remains unfulfilled. Acute wounds (simple and complex) may also benefit from accelerated healing, reduced scar formation, decreased complications, improved esthetic appearance and overall tactility and durability. Wound healing may be addressed with a variety of DKK1 inhibition applications by using an anti-DKK1 agent and/or other promoter(s) of Wnt signaling. In the case of diabetic ulcers, there are few good treatments for this chronic wound and sustained closure is difficult to achieve. Development of a combination product including an anti-DKK1 agent and/or other promoter(s) of Wnt signaling plus PRT or other ABMs, may create a more effective barrier membrane with a combined biologic agent for tissue regeneration. In the case of burns or scar tissue, the healing cascade can be stimulated with the combination of an anti-DKK1 agent or other promoter of Wnt signaling pathway, PRT and ABMs to rejuvenate and regenerate the damaged tissue.

AGA and other causes of alopecia are common, and treatments are varied, with mixed success. In the case of cancer related hair loss or radiation induced wound or hair problems, improved therapies are needed. Optimal treatment for wounds and hair loss may involve a targeted therapeutic option which combines therapies such as an anti-DKK1 agent and/or other promoter(s) of Wnt signaling pathway and PRTs. In addition, the desire to grow hair where none previously existed to enhance the appearance of facial hair, such as moustaches and beards, is another potential esthetic application for an anti-DKK1 agent and/or other promoter(s) of Wnt signaling plus PRT plus an ABM (graft).

Similarly, alopecia due to various causes may also be treated with the composition described herein by restoring senescent hair follicles, developing new hair follicles, and increasing hair shaft formation and thickness.

In some therapeutic applications it may be desirable to remove inhibitors of Wnt signaling such as DKK1 from the ABM material such as by filtering or stripping or other approaches prior to administering the ABM to the patient. The therapeutic ABM maybe treated at various steps of its ex vivo preparation, or after the ABM is fully prepared. In order to remove DKK1, for example, the ABM may be passed through an apparatus that acts as a filter, matrix, chamber, or labyrinth. This apparatus could contain various filtering mechanisms or coatings or other mechanical or electrical or magnetic devices such as vibratory or current or magnetic methods to separate and propel or otherwise propagate the ABM through the apparatus. In such an apparatus, the exogenous anti-DKK1 agent or other agent that binds another inhibitor of Wnt signaling is adherent or otherwise attached to or confined within surfaces or components found within the apparatus. As the ABM passes through the apparatus, DKK1 will be bound by the anti-DKK1 agent and stripped (extracted) from the expressed ABM, thereby preparing a “DKK1 Depleted” ABM that can be administered to the patient. As such, an aspect of the method provides preparing a composition for administering to a subject to treat bone defect, wound healing or hair growth, which includes obtaining an ABM from the subject, removing at least 50%, 55%, 60%, 65%, 70%, 75%, 80%, 85%, 90%, or 95% of DKK1 or another Wnt antagonist from the ABM, and subsequently adding an anti-DKK1 antibody or another promoter of Wnt signaling to the ABM to form the composition for administration. an ABM or non-autologous tissue which has been treated or prepared to have at least 50%, 55%, 60%, 65%, 70%, 75%, 80%, 85%, 90%, or 95% of DKK1 or another Wnt antagonist removed.

In one preferred method a DKK1-Depleted Serum (DDS) is created, which comprises of blood-derived serum from which DKK1 has been physically depleted with modest or no effects on other constituents of the serum. Serum is a preferred ABM for this application based on the coagulation step involved in its preparation, which provokes platelets to release much of their granule contents such that growth factor levels are maximal and DKK1 is bioavailable for depletion approaches, along with its cell-free nature, which is compatible with sterile filtration of the final product. The method for producing DDS is accomplished by executing the following steps. Modifications and alterations to the system may be made to improve processing and reduce time which encompass further embodiments. An agent that physically binds to DKK1 with sufficiently high affinity and avidity and specificity, such as a large molecule or protein or antibody, is conjugated to magnetic polystyrene beads or a functional equivalent. The beads provide a high surface-to-volume ratio to facilitate DKK1 binding, and their magnetic property facilitates their removal in latter steps of this process. A clinician, nurse or technician skilled in performing venipuncture performs a standard blood draw and collects the blood into the serum separator tubes, followed by standard procedures to generate serum. The serum is then transferred to tubes or other vessel containing magnetic DKAB beads. The bead-serum mixture is incubated for at least 15 minutes or for up to an overnight incubation, during which time DKK1 becomes bound to the beads. A magnet is then applied to an outer surface of the vessel or container to pull the beads aside, allowing the remaining serum to be easily removed from the tube using a blunt needle and syringe. The blunt needle is then swapped for a luer-lock syringe filter through which the serum is then passed, which will filter out any beads that may not be bound to the magnet. This filter, or a series of filters with decreasing pore sizes (for example, a 1-micron filter to eliminate residual cells and beads followed by a 0.22-0.50 micron filter to eliminate bacteria), will also clarify and sterilize the serum, with the serum discharged into a collection vessel. The final DDS material is now ready for local injection into or application onto the patient. DDS that is not used for the current treatment may be reserved for future treatment sessions, in many cases via storage in a refrigerator or freezer to better maintain its sterility. The DKK1 depletion system comprises the following features; magnetic beads coated with DKAB or other DKK1-specific binding agent, blood collection tubes for centrifugation, transfers and incubation vessels, a magnet, standard syringes, syringe filter(s), and needle for blood collection. The method of physically extracting and depleting DKK1 from an ABM has been developed whereby multiple parameters of the aforementioned system; bead quantity, incubation time, and agitation have been examined. Agitation on an orbital shaker platform had no effect on the system performance (data not shown), while increased quantity of beads and increased incubation time improved DKK1 depletion performance. The feasibility of this system is demonstrated in FIG. 4, wherein DKAB-coated magnetic beads were used to reduce soluble (extracellular) DKK1 levels by 85% while having only modest effects on soluble concentrations of VEGF and PDGF. Furthermore, the data in FIG. 5 demonstrates significant DKK1 depletion can be achieved with 15 minutes of incubation and depletion is further improved at 30 and 60 minutes.

### Mechanisms

It is proposed herein that certain ABMs will contain high levels of DKK1 that are released before, during, or after the ABM is prepared, which will limit the benefits of those ABMs in treating certain conditions including bone injuries, wounds, and hair loss. One of the most commonly used platelet-containing ABMs is PRP, a typically autologous therapy prepared from the patients' own blood by removing or reducing the volume of red cell cells and platelet-poor plasma (PPP), which delivers a reduced volume of plasma with a higher concentration of platelets and, in some cases, leukocytes as well. Various embodiments provide that PRP has enriched levels of numerous platelet-released growth factors and cytokines, which underlie the pro-healing benefits of that ABM, including PDGF, VEGF, IGF-I, TGF-β, BMP-2, and others. These factors are released from platelets in a bioavailable form, particularly when platelets become activated, whether by ex vivo exposure of platelets to exogenous platelet-activating factors such as thrombin and calcium chloride, or by in situ platelet exposure to endogenous platelet-activating factors at the site where PRP is delivered, such as collagen, thrombin, and tissue factor. Some studies show that locally-applied PRP in various forms increases local bone formation and regeneration in craniomaxillofacial models, but in general, alveolar bone augmentation and regeneration with PRP is inconsistent and modest at best. PRP has been shown to increase bone volume and arthrodesis in some rodent spinal fusion studies but not in others, and the bone-augmenting effects of PRP in spinal fusion and other orthopedic applications are generally modest and inconsistent. The minimal bone-augmenting effects of PRP contrasts with the established bone-augmenting effects of many of its constituent growth factors, as summarized above. This apparent discordance is consistent with the novel hypothesis that a high level of platelet-derived DKK1 exists in PRP (and other platelet-enriched ABMs) that diminishes the therapeutic potential of PRP in bone, skin, and hair applications. Data from several studies indirectly support this hypothesis: 1) platelet-poor plasma (PPP) can be more efficacious than PRP in stimulating bone formation and alveolar bone regeneration; 2) PRTs with very high platelet concentrations can cause relatively lesser stimulation of osteoblasts, lesser proliferation of endothelial cells, and lesser migration of epithelial cells and vascular pericytes compared with PRP containing lower platelet concentrations; and 3) PRP prepared with the highest relative platelet concentration can inhibit rather than promote bone regeneration around implants, a result described as ‘paradoxical’ and ‘astonishing’. Some investigators suggest that a lack of osteogenic responses to PRTs may relate to excessive levels or premature release of various platelet-derived growth factors, but this hypothesis is unlikely based on the relatively modest concentrations of growth factors found in even the mostly highly platelet-enriched PRTs. Other investigators propose that these paradoxical effects relate to the use of certain pro-coagulation factors or other variables involved in preparing certain PRTs. But no investigators have previously demonstrated or considered that PRTs may have high levels of DKK1 that interfere with certain regenerative processes, including bone repair, wound healing, and hair restoration.

Several clinical studies show that patients with fresh fractures have elevated circulating DKK1 levels, yet no investigators proposed or speculated that this elevation may relate to DKK1 release from platelets that become activated due to fracture, or that this source of DKK1 may inhibit the rate or degree of bone repair.

DKK1 released from activated platelets influences inflammatory reactions between platelets and endothelial cells in atherosclerosis, indicating that platelet-derived DKK1 is biologically active and functional. DKK1 release from activated platelets likely occurs after skeletal injury, and many orthopedic and dental procedures induce substantial bleeding and or hematoma formation. Whether due to injury, damage, or medical/surgical procedure, bleeding and hematoma are likely to increase local DKK1 levels in ways that may blunt the osteogenic and regenerative effects of these otherwise favorable responses. We propose that platelet secretion of DKK1 contributes to this suboptimal milieu. Similarly, many surgical procedures, including spinal fusion, alveolar bone augmentation, microfracture surgery, and hair restoration involve decortication, or micro-needling, or other approaches that deliberately induce the localized expression of fresh blood, which leads to the release from platelets of growth factors and other substances that trigger therapeutically important tissue responses. In each of these and other examples, platelet activation seems equally likely to trigger their release of DKK1, which stands to diminish the regenerative benefits of the procedure.

A major limitation of distraction osteogenesis is the prolonged time patients must remain in a cumbersome external fixator while new bone forms and consolidates. DKK1 is not expressed during the post-osteotomy latency (i.e. resting) phase of DO, but DKK1 expression is upregulated during the distraction and consolidation phases of the procedure, which may control (i.e. limit) the rate and extent of new bone formation.

While DKK1 levels had never before been evaluated in PRTs, several studies demonstrate the PRTs that are rich in platelet-released growth factors failed to improve distraction osteogenesis, which may relate in part to high levels of platelet-derived DKK1 in the tested PRTs.

Platelet-containing ABMs used to promote wound healing may also benefit from the addition or incorporation of therapeutic agents that promote Wnt signaling. Increased Wnt signaling induced by topical LiCl promotes dermal wound healing in rodents, whereas recombinant DKK1 delayed wound healing. Those findings suggest that high DKK1 levels in ABMs that contain platelets or platelet-released growth factors may limit the healing potential of those ABMs. Consistent with that possibility, platelet releasate dose-dependently inhibited keratinocyte proliferation, an effect that could not be attributed to excessive levels of various platelet-released growth factors and cytokines, based on evidence that their inhibition via neutralizing antibodies did not reverse this inhibitory effect. We hypothesize that this anti-proliferative effect may be due to high DKK1 levels in the platelet releasate, which is supported by other evidence that reduced DKK1 expression in mice increases epithelial cell proliferation. Other studies show that activated platelet-rich plasma supernatant strongly stimulates endothelial cell proliferation and invasion, but only when platelet concentrations were moderate. There was a clear reversal of efficacy at higher levels of platelet enrichment, which the authors attributed to potentially excessive levels of growth factors that had counter-productive effects on wound healing. These and other findings of suboptimal therapeutic effects of ‘hyper-enriched’ PRTs led to proposals, now widely accepted, that platelet concentrations in PRTs should not exceed around 1 million per microliter. Yet it is unlikely that platelet-released growth factors in hyper-enriched PRTs are so high as to cause a reversal of therapeutic effects; for example, recombinant PDGF-BB is efficacious for local bone augmentation when delivered at levels that exceed by >100-fold the maximum achievable levels in PRTs. It seems more biologically plausible that excessive levels of DKK1 that come with increasing degrees of platelet enrichment could contribute to a reversal of therapeutic responses to PRTs. In the case of wound healing, animal studies involving DKK1 knock-down indicate that DKK1 inhibits the migration of epithelial cells, and DKK1 also inhibits the migration of vascular pericytes, including that which is stimulated by PDGF. The neutralization or removal of DKK1 from PRTs and certain other ABMs may unleash previously unappreciated therapeutic benefits of platelet hyper-enrichment, with hyper-enriched growth factors able to better promote healing due to the absence of excessive DKK1 levels. Alternatively, or additionally, agents that promote Wnt signaling via mechanisms other than DKK1 inhibition may be added to PRTs and certain other ABMs to overcome or compensate for DKK1-mediated inhibition of Wnt signaling, thereby enhancing the regenerative milieu of the ABM and the site of its application.

Wnt signaling is also important for hair follicle viability and vitality. Androgens upregulate DKK1 expression within hair follicles, a result that aligns with data indicating that DKK1 is an important mediator of androgenic alopecia and alopecia areata. It is therefore surprising that while PRTs are increasingly used to treat hair loss or insufficiency, no investigators have reported DKK1 levels in PRTs, and none has suggested that the efficacy of PRTs for hair loss, wound healing, or bone augmentation may be limited by DKK1 released from platelets during or after PRT preparation. By supplementing platelet-containing ABMs with an anti-DKK1 agent, the untoward effects of platelet-derived DKK1 may be eliminated or reduced while preserving the therapeutic benefits of various platelet-secreted growth factors, thereby amplifying the therapeutic benefits of the ABM for hair growth and wound healing. Alternatively, or additionally, agents that promote Wnt signaling via mechanisms other than DKK1 inhibition may be added to PRTs and certain other ABMs to overcome or compensate for the adverse effects of high DKK1 levels, thereby enhancing Wnt-mediated follicle-stimulating effects of the ABM.

Pluripotent stem cells have the potential to differentiate along various cell lineages depending on their origin and the milieu in which they reside or are delivered. Such stem cells are increasingly used as ABMs to promote bone formation and wound healing, and evidence suggests that bioavailable Wnts and Wnt signaling are important cues for encouraging stem cells to differentiate along lineages that promote bone formation and wound healing. The addition of therapeutic agents that promote Wnt signaling to stem cell therapies may therefore enhance the ability of stem cell-based ABMs to promote bone formation and wound healing.

Bone marrow is the birth place of platelets, and when stem cells are harvested from bone marrow by a needle, trocar, reamer, aspirator, or other device, local platelets are likely to become activated, causing them to release growth factors and DKK1 that can influence the differentiation of stem cells captured by these procedures. DKK1 is known to inhibit the osteogenic differentiation of mesenchymal stem cells (MSCs), but no investigators have proposed that the act of harvesting MSCs from bone marrow may inadvertently expose MSCs to DKK1 released from activated platelets. The preparation of BMAC from BMA lead to substantial platelet enrichment, which should lead to higher DKK1 levels compared with BMA due to spontaneous or incidental platelet activation. This DKK1 stands to limit the osteogenic differentiation of stem cells within the ABM itself, and at the site where the ABM is delivered. The addition of an anti-DKK1 agent or other promoter of Wnt signaling would overcome the inhibitory effects of platelet-derived DKK1, thereby increasing the vigor or proportion of stem cells that differentiate along the osteogenic lineage, thereby improve the efficacy of the ABM. This notion is supported by evidence that even modestly-elevated DKK1 levels in the bone marrow of patients with multiple myeloma (i.e., 50% to 3.5-fold above levels in healthy control subjects) were sufficient to impair the osteogenic differentiation of MSCs.

Some ABMs, including fibrin gel, PRF, and clotted blood, have a gel-like form factor imparted by fibrin formation and cross-linking, creating a matrix with several potential therapeutic benefits. These gel-like matrices have the potential to bind tissues together to arrest bleeding and promote wound approximation and healing, and may also provide a degradable provisional matrix upon which resident cells can act to reconstitute tissue. The cohesive properties of some gel-based ABMs can also be exploited to help bind and contain grafting materials, thereby improving their physical handling characteristics and limiting their undesirable leakage or migration from the graft recipient site. Gel-based ABMs can also confer sustained release of endogenous growth factors they may contain, as well as of therapeutic agents that promote Wnt signaling. Considering that autologous fibrin-based ABMs are often used in dentistry, maxillofacial surgery, orthopedic surgery, plastic surgery, and esthetics, and considering that DKK1 secretion from platelets may exerts untoward effects at the very sites where autologous fibrin-based ABMs are often applied, there is a unique and therapeutically rational opportunity to add an anti-DKK1 agent or other promoter of Wnt signaling to fibrin-based ABMs in certain therapeutic settings.

Some of the aforementioned examples highlight the potential for increasing Wnt signaling and promoting tissue regeneration or augmentation by adding various therapeutic agents to ABMs, both of which are delivered to or come together within the patient. This invention also proposes that the physical elimination of DKK1 from an ABM prior to the ABM's administration to the patient may also improve the ability of the ABM to promote tissue regeneration or augmentation. A variety of methods could effectively reduce DKK1 levels in harvested autologous tissue before, during, or after said autologous tissue is processed into or is delivered as an ABM. For example, DKK1 binding proteins (including but not limited to DKAB) may be attached otherwise immobilized to surfaces within collection chambers, or tissue processing vessels, or a labyrinth, or matrix, or other apparatus. By this or other approaches, DKK1 within the ABM or ABM starting material would attach to the DKK1 binding protein before, during, or after the ABM preparation steps, with the DKK1 and its binding protein left behind in the apparatus while the remaining material is retrieved or collected or otherwise passed into or upon the patient. In other embodiments, DKK1-binding proteins such as but not limited to DKAB may be immobilized to beads or other solid materials that provide a high surface-to-volume ratio, to which autologous DKK1 would attach and be left behind as those solid materials are physically separated from the remaining autologous tissue before, during, or after its processing into an ABM. By these or other methods, growth factors, cytokines, and other ABM-derived factors with potentially desirable therapeutic properties can be administered without the negative effects of high DKK1 levels in the ABM, and without administering therapeutically meaningful amounts of the DKK1-binding protein to the patient.

Whether by itself or in conjunction with an exogenous platelet activator, the injection of an anti-DKK1 agent and/or other promoter(s) of Wnt signaling directly into an in-situ fracture hematoma or other procedure site immediately places the therapeutic agent at the site of injury or surgery, which may be clinically important based on the transient nature of DKK1 expression in those settings. This transience has multiple biological bases: 1) injuries and surgeries create a transiently-activated platelet population that typically ends within several hours to several days, 2) platelet activation leads to full release of their stored DKK1 (and beneficial growth factors) within minutes, and 3) the elimination half-life of DKK1 is less than 30 minutes. These phenomena suggest the existence of a finite and potentially brief window for optimal intervention with an anti-DKK1 agent or other promoter of Wnt signaling, which may not be achieved if those therapeutic agents were delivered as a standard subcutaneous injection at a remote location such as the abdomen or upper thigh. Subcutaneous injection-based delivery of certain therapeutic agents, including antibodies and other large molecules, may have significant limitations when the goal is to mitigate the adverse influence of acutely elevated DKK1 levels. For one, subcutaneous delivery often leads to a delay of several days in achieving peak blood levels, leading to a commensurate delay in the attainment of maximum drug levels at local sites of interest. Furthermore, subcutaneous delivery can significantly reduce the bioavailability of certain injected agent leading to lower maximum and overall drug exposure. Such delays and bioavailability losses may allow greater amounts of acutely released DKK1 to impair Wnt signaling, leading to suboptimal therapeutic responses to the injected agent. Some of those limitations may be minimized by delivering the anti-DKK1 agent or other promoter of Wnt signaling intravenously, but there are many clinical settings where intravenous drug delivery is challenging, inconvenient, cost-prohibitive, or otherwise impractical. Furthermore, intravenous delivery is unlikely to create the very high local drug levels that are achieved via the local injection of the same amount of the agent, whether in conjunction with a harvested ABM that is delivered to a site of damage or surgery, or directly into an ABM that remains within the patient, such as a fracture hematoma.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1: Anti-DKK1 Agent (DKAB) Combined with Platelet-Rich Plasma (DKAB-PRP) as an Adjuvant for Bone Fracture Repair or Osteotomy Healing

A patient's peripheral blood is drawn and PRP is prepared using commercially available systems, which may include a custom kit that includes DKAB. DKAB from the kit or from a separate source is added to the PRP and mixed. The combined DKAB-PRP product can then be applied to the fracture or osteotomy site by physical transfer, or adding to a spraying apparatus, or painting via a surgical instrument, or other means. Bone fixation and/or stabilization is then completed using accepted orthopedic protocols. The DKAB-PRP is expected to promote chondrogenesis and osteogenesis at the fracture site by virtue of DKAB-mediated inhibition of DKK1 within the PRP and within the site of administration, and growth factors provided by the PRP, leading to improved bridging and stability. The DKAB-PRP may also promote osteogenesis on and around fixation hardware used to provide early stabilization of the healing fracture or osteotomy, leading to a further enhancement in fracture stability.

### Example 2: Promoter of Wnt Signaling (WAY-316606) Combined with Bone Marrow Aspirate Concentrate (BMAC) for Percutaneous Treatment of Nonunion

Bone marrow aspirate (BMA) is harvested from the iliac crest of an anesthetized patient with a non-union or delayed-union fracture at the time of corrective surgery. The BMA is concentrated into BMAC using on-label techniques from commercially available BMAC systems. WAY-316606, a small molecule that promotes Wnt signaling by inhibiting the Wnt antagonist sFRP-1, is added and mixed into the BMAC to form WAY-316606-BMAC. A percutaneous or minimally-invasive approach is used to access the bone nonunion site on the body using a commercially available delivery cannula apparatus with or without imaging guidance. The local bone site is abraded using standard orthopaedic instrumentation such as a burr or rasp to cause decortication and local bone bleeding. The WAY-316606-BMAC product is injected via the apparatus directly to the intended target site using accepted orthopaedic principles. The BMAC provides mesenchymal stem cells (MSCs) with the capability of differentiating into bone-forming osteoblasts. These MSCs will be exposed to numerous endogenous growth factors released locally from platelets as a result of the surgical procedure, including the decortication, which will encourage their osteogenic differentiation and/or activity. Platelet activation in response to surgery will also release DKK1, and WAY-316606 will at least partially overcome the inhibitory effects of DKK1 on Wnt signaling by inhibiting sFRP1, a different Wnt antagonist that limits Wnt signaling. sFRP1 inhibition by WAY-316606 will thereby promote the osteogenic differentiation of BMAC-associated MSCs and will also promote bone formation by stimulating Wnt signaling in resident osteoblasts that would otherwise be under the osteogenesis-inhibiting influence of sFPR1 and DKK1.

### Example 3: Promoter of Wnt Signaling (Lithium) Combined with Bone Marrow Aspirate Concentrate (BMAC) for Treatment of Avascular Necrosis (AVN) of Femoral Head

A patient with avascular necrosis of the femoral head undergoes decompression of the femoral head surgery, whereby multiple holes are drilled into the femoral head. During the same procedure, bone marrow is aspirated from the patient's iliac crest and processed into BMAC. A lithium salt such as lithium chloride or lithium carbonate is added to the BMAC mixture and mixed, then transferred or drawn into a syringe or other type of injector device. The femoral head is drilled using standard orthopaedic instrumentation to cause decortication and local bone bleeding. The lithium-BMAC product is injected directly through the drill holes into the necrotic site using accepted orthopaedic principles. The lithium-BMAC product is expected to increase the osteogenic differentiation of stem cells within the injected BMAC via their exposure to LiCl, which promotes Wnt signaling. The lithium-BMAC product will also promote osteogenesis by the effects of lithium ion Wnt signaling in resident osteoblasts and osteogenic stem cells in the surrounding bone. This stimulation of osteogenesis is expected to enhance or accelerate new bone formation and thereby promote structural restoration, leading to faster recovery and improved hip function. These approaches have the advantage over systemically-administered LiCl because the systemically-administered drug may not reach avascular sites in appreciable amounts due to the lack of blood supply. Locally-administered lithium-BMAC may also limit some of the potentially adverse effects of lithium, including psychoactive effects.

### Example 4: Anti-Sclerostin Agent (Sclerostin Antibody; Scl-AB) Combined with Platelet-Rich Plasma (Scl-Ab-PRP) for Bone Interference Screw Fixation as an Adjuvant for Anterior Cruciate Ligament (ACL) Reconstruction

An ACL reconstruction procedure is followed per current orthopaedic principles, with bone tunnels for the graft prepared according to surgeon preference and implants. Peripheral blood is drawn from the patient and used to prepare PRP. Scl-Ab is added to the PRP and combined within the surgical field or other clinical setting. The Scl-Ab-PRP is delivered to previously prepared bone tunnels with a syringe or other applicator device, and the standard ACL procedure is then followed by placing the patellar bone-tendon-bone graft into the tunnels, followed by the placement of an interference screw and completion of the ACL reconstruction procedure. Scl-Ab-PRP is expected to promote bone formation by the combined effects of PRP-derived growth factors and the Scl-Ab. The Scl-Ab is expected to increase Wnt signaling by compensating for the inhibitory effects of high DKK1 present in the PRP and that which is secreted by activated platelets and perturbed osteocytes at the surgical site. These effects are expected to promote bone growth from the bone tunnel walls or neighboring bone surfaces and onto and around the interference screw, and between the bone-tendon-bone graft and the bone tunnel, leading to enhanced or accelerated healing that increases the stability of the reconstruction.

### Example 5: Multiple (Repeated) Treatments

The patient from Example 4 visits his or her orthopedic surgeon at a post-surgical follow-up meeting, during which time a second injection of Scl-Ab-PRP is delivered percutaneously to the region of the surgically-repaired ACL. This second treatment is expected to further promote or sustain osteogenesis at the repaired site through the mechanisms described in Example 10.

### Example 6: Promoter of Wnt Signaling (Wnt3a) Combined with Reamer-Irrigator Aspirate (RIA) as an Adjuvant for Repair of Long Bone Fractures with Intramedullary Nailing

A patient with a femoral shaft fracture undergoes harvesting of bone graft material via reamer-irrigator aspirate (MA) harvested per orthopedic protocols. The MA is prepared as per orthopedic protocol, and the RIA is then mixed with liposomally-formulated Wnt3a and placed into the femoral defect. Based on surgeon preference and procedure applicability, bone graft substitutes may also be added to the mixture to enhance the graft volume. An intramedullary nail prosthesis is then inserted into the femoral canal and fixated per orthopedic protocols. Additional Wnt3a-RIA is applied around the prosthesis to further enhance bone formation and stability of the device. The Wnt3a-RIA product is expected to increase the osteogenic differentiation of stem cells delivered in the RIA as well as resident stem cells in the vicinity of the surgical site. RIA has been shown to be rich in several platelet-released growth factors, which may also promote bone formation by osteoblasts in the fractured bone. The Wnt pathway-stimulating effects of the liposomal Wnt3a is expected to overcome the pathway-inhibiting effects of high DKK1 in the RIA, based on the evidence that RIA is rich in other platelet-released factors. These myriad sources of osteogenic stimuli are expected to enhance or accelerate fixation of the prosthesis to the bone by promoting new bone formation on and around the prosthesis.

### Example 7: Promoter of Wnt Signaling (LY2090314) Combined with Autologous Plasma Gel Plus Calcium Phosphate Granules as a Bone Void Filler

Blood is drawn from the patient and used to prepare normal plasma, to which LY2090314 is added and mixed. LY2090314 is a small molecule that promotes Wnt signaling by inhibiting GSK-30. The LY2090314-containing plasma is then activated by the addition of pro-coagulation factors such as thrombin and/or calcium chloride. Once the LY2090314-plasma polymerizes into a fibrin-based gel, the material is added to calcium-phosphate granules to create a cohesive slurry or mixture. This mixture is placed into a bone void and the surgical procedure completed according to orthopedic protocol. The addition of LY2090314-plasma gel to the calcium phosphate granules enhances their handing properties and promotes containment of the granules to minimize their migration out of the void, with the granules serving as an osteoconductive substrate to accelerate refilling of the void via new bone formation. The LY2090314-plasma gel is expected to promote osteogenesis via responses of resident bone cells and mesenchymal stem cells to LY2090314 and growth factors that are gradually released from the plasma gel via its fibrinolysis. LY2090314 enters those cells and promotes Wnt signaling by stabilizing beta-catenin, which will at least partially override the inhibitory effects of platelet-derived DKK1 that is secreted into the plasma gel in reaction to the plasma activation step. Furthermore, growth factors embedded within and released from the plasma gel will promote bone formation by stimulating growth factor receptors, which trigger signaling pathways that are partially, largely, or fully independent of Wnt signaling.

### Example 8: Anti-DKK1 Agent (DKAB) Combined with Autologous Bone Graft as an Adjuvant for Spinal Fusion

Autologous bone graft is harvested from the spine and/or iliac crest of a patient undergoing a spinal fusion procedure. DKAB is added to the autograft material and mixed, with or without additional allogeneic or synthetic bone graft materials that may be included to extend the volume of graft material. Regions of the spine where arthrodesis is desired are prepared by osteotomy, decortication, and/or endplate preparation as per spinal surgery protocols. The DKAB-Graft product is placed within the graft window or graft chamber of a bone allograft ring, or fusion spacer, or fusion cage, which is then inserted into the interbody space of the spine per spinal surgery protocols. Additional DKAB-Graft may be added to the interbody space around the ring/spacer/cage device and around posterolateral spinal elements to further promote intersegmental arthrodesis. DKAB-Graft is expected to enhance osseointegration of the fusion device and arthrodesis across the interbody segment at decorticated or prepared or grafted spinal elements by neutralizing DKK1 from multiple sources, including DKK1 within the autograft, DKK1 secreted by hematogenous platelets arriving at bleeding sites, and DKK1 released from perturbed osteocytes at sites of osteotomy, decortication, and endplate preparation. Increased bone regeneration within the spine is expected to enhance or accelerate the stability of the fused spinal segments and improve the patient's pain and functioning.

### Example 9: Promoter of Wnt Signaling (R-Spondin) Combined with Surgical Blood as an Adjuvant for Spinal Fusion

Whole blood is collected from the surgical site of a patient undergoing spinal fusion surgery. This salvaged blood is mixed with recombinant R-Spondin, a potentiator of Wnt signaling, and the blood is allowed to coagulate and clot. The R-Spondin-clot mixture is then placed or pressed into the graft chamber, pores, or other openings in a spinal fusion cage. The fusion cage containing the R-Spondin-clot product can then be placed into the spine region requiring fusion, and the surgical procedure completed per orthopedic protocol. R-Spondin plus coagulated blood is expected to promote osseointegration between the fusion cage and native bone, and to promote the fusion of adjacent vertebral segments, thereby achieving enhanced or accelerated stabilization of the problematic spinal region. These benefits are expected to derive from the ability of RSpondin to potentiate Wnt signaling arising from the activity of endogenous Wnts that manage to bind and activate their receptors (LRGs) despite elevated levels of DKK1 released by activated platelets in the surgical blood and the surgical site and DKK1 released from perturbed osteocytes at the surgical site. Concomitantly, the fibrin matrix component of the clot component of the product will provide slow, sustained release of platelet-released growth factors as well as the R-Spondin, thereby creating a more favorable osteogenic milieu for several hours or days after the spinal fusion surgery is completed.

### Example 10: Promoter of Wnt Signaling (Wnt Surrogate) Combined with Platelet-Rich Fibrin (PRF) Plus Calcium-Phosphate (CaP) Granules for Maxillofacial Bone Augmentation

A patient undergoing maxillofacial bone augmentation for esthetic purposes undergoes a peripheral blood draw from which PRP is prepared. A Wnt surrogate molecule such as scFV-DKK1c, which acts by inducing heterodimerization of Wnt receptors (FZD and LRP5/6), is added to the PRP and mixed within the surgical field or clinical setting. The scFV-DKK1c-PRP is then allowed or induced to coagulate and clot to create cross-linked fibrin, resulting in scFV-DKK1c-PRF that can then be added to calcium-phosphate (CaP) granules and mixed into a cohesive slurry, which would be applied subcutaneously or sub-periosteally in maxillofacial facial regions where augmentation is desired. This therapeutic approach is expected to increase maxillofacial and skin projection or to otherwise enhance maxillofacial esthetics, including that which is desirable in patients with maxillofacial aging, trauma, tumor resections, or developmental defects. This combination product is expected to increase bone formation and maxillofacial bone volume via growth factors released from the PRF and via increased Wnt signaling via the effects of scFV-DKK1c. scFV-DKK1c will overcome to a meaningful degree the deleterious effects of high DKK1 levels in the PRF by displacing or competing with DKK1 binding to Wnt receptors. The PRF also provides desirable cohesiveness for the placement and retention of graft particles at the site of interest, thereby minimizing their migration beyond the site of implantation. Such migration is currently minimized in esthetic applications by placing CaP granules subperiosteally, but the addition of a Wnt surrogate and PRF (or other clotted or gel-like ABMs) may allow for the safe and efficacious placement of space-filling CaP granules or other bone graft substitutes subcutaneously, which may reduce morbidity of the procedure and accelerate recovery times and improvements to esthetics.

### Example 11: Anti-DKK1 Agent (DKAB) Combined with Platelet Rich Fibrin (PRF) Spray Plus Bone Graft for Alveolar Bone Augmentation

Blood is drawn from a patient who requires alveolar bone augmentation for the placement of dental implants. The blood is processed into PRP and DKAB is added to the PRP. The DKAB-PRP is drawn into one chamber of a dual-chamber syringe system, with the other chamber containing coagulation-inducing factors such as thrombin and/or calcium chloride. Meanwhile, an autologous bone graft or graft substitute is applied to the decorticated or otherwise surgically prepared alveolar bone site in need of volume augmentation. The dual-chamber syringe is placed over the bone graft and the plunger pressed, leading to the mixing of DKAB-PRP with the coagulation-inducing factors, with the solution emerging through a single spray nozzle as a rapidly-forming fibrin gel that coats the bone graft and surgical site. The grafted surgical site is then closed via apposition and suturing of the overlying gingiva, and the surgical procedure is completed using dental surgery protocols. The addition of DKAB-PRF to the bone graft is expected to enhance local bone formation via the inhibitory effect of DKAB on DKK1 released by platelets within the administered PRF, and DKK1 released by resident platelets arriving at the surgical site, and DKK1 released locally from perturbed osteocytes. Increased bone formation by these means enhances or accelerates osseointegration of the bone graft to the native alveolar bone bed, leading to shorter treatment times for the placement of implants and their functional loading. The PRF will also serve as a provisional matrix that supports the ingrowth of vascular and osteogenic cells.

### Example 12: Anti-DKK1 Agent (DKAB) Combined with Platelet-Rich Plasma (PRP) as an Adjuvant for Chronic Ulcer or Pressure Sore Healing in Diabetic Patients

A patient's peripheral blood is drawn by venipuncture and PRP is prepared using a commercially available kit, which may include a custom kit with which DKAB is included. DKAB is added to the PRP and mixed, and the DKAB-PRP is then be applied topically to the chronic wound ulceration site by physical transfer, spraying, painting, or other means that may include an additional wound cover/closure device. Wound treatment is followed otherwise using standard of practice wound care protocols. The DKAB-PRP is expected to promote cellular growth and tissue healing by the effects of PRP-derived growth factors plus the inhibitory effects of DKAB on DKK1 released from platelets within the PRP and the resident platelets within and around the wound site. As the PRP becomes activated by contact with wound tissue, it will convert to a fibrin gel that provides improved wound coverage and barrier formation, and will also undergo additional platelet release of regenerative growth factors. These combined effects will further improve tissue bridging, epithelialization, and wound closure.

### Example 13: Anti-DKK1 (DKAB) Combined with Platelet-Rich Plasma (PRP) Plus Micronized Adipose Tissue (MAT) as Treatment for Partial or Full Thickness Burns

A patient's peripheral blood is drawn and PRP is prepared using a proprietary custom kit or a commercially available PRP system. In addition, an adipose harvesting and processing system is employed for adipose tissue extraction and is further processed into a micronized adipose tissue (MAT) or is enzymatically digested into a stromal-vascular fraction (SVF). DKAB is added to the PRP and combined with the processed adipose tissue using a surgical mixing apparatus such as a syringe transfer device, filter, or other closed system method known in the art. The DKAB-PRP-MAT product is applied to the partial or full thickness burn site by physical transfer, spraying, painting, or other means that may include an additional wound cover/closure device. Wound treatment is followed otherwise using standard of practice wound care protocols in burn patients as in a burn center or wound care clinic. The DKAB-PRP-MAT is expected to promote cellular growth and tissue healing through multiple effects, including release of regenerative growth factors from PRP-associated platelets, DKAB-mediated inhibition of PRP-associated DKK1, release of MSCs from MAT as well as physical barrier protection from the adipose fiber material and PRP-associated fibrin. These combined effects will further improve tissue bridging, epithelialization, wound closure and tissue regeneration, leading to improved wound closure or epithelialization and barrier formation or skin coloration and rejuvenation.

### Example 14: Anti-DKK1 Agent (DKAB) Combined with PRP (PRP) for Hair Growth

A patient with hair loss is undergoing a procedure for hair restoration to receive PRP injections in multiple locations in the scalp. PRP is prepared from blood obtained from the patient by venipuncture and DKAB is added to the PRP and mixed. The DKAB-PRP product is injected into the scalp similar to how normal PRP is applied, which is expected to promote local hair follicle development, shaft growth and regrowth by the combined effects of PRP-derived growth factors and the inhibitory effects of DKAB on DKK1 in the PRP itself and that which is secreted by activated platelets in response to the injections, and that which is secreted by hair follicle cells in response to endogenous androgens such as dihydrotestosterone.

### Example 15: Promoter of Wnt Signaling (WAY-316606) Combined with Autologous Platelet-Rich Plasma (PRP) as an Adjuvant to Hair Grafting

A patient with alopecia is undergoing hair grafting. WAY-316606, a small molecule that promotes Wnt signaling by inhibiting the Wnt antagonist sFPR-1, is added to autologous PRP and mixed. Hair grafts harvested from the patient are immersed, coated, or otherwise exposed to the WAY-316606-PRP product for 15 minutes followed by surgical implantation of the hair grafts into desired locations in the scalp as per normal procedures. Stored aliquots of the WAY-316606-PRP product can also be injected around the graft recipient sites during subsequent follow-up visits to further promote graft viability and vitality. The administration of WAY-316606-PRP is expected to favor the survival and vitality of hair grafts via the combined effects of PRP-derived growth factors and WAY-316606-induced promotion of Wnt signaling by neutralizing sFPR-1, which will at least partially overcome the inhibitory effects of high DKK1 within the PRP and that which may be secreted by activated platelets during graft placement.

### Example 16: Apparatus that Physically Depletes Serum or Other Aqueous ABMs of DKK1 or Other Inhibitors of Wnt Signaling to Enhance their Efficacy for Hair Restoration

A patient's peripheral blood is drawn by venipuncture and serum is prepared using a serum separator tube. The serum is then temporarily incubated in an apparatus that acts much like a filter or labyrinth to physically remove (‘strip’) DKK1 from the PRP. In such an apparatus, an exogenous anti-DKK1 agent such as DKAB, or other agents that bind other Wnt antagonists, including but not limited to sclerostin, sFRP-1 and other sFRPs, WIF-1, and/or Wise/SOSTDC, is adherent or otherwise attached to or confined within surfaces or components or spaces found within the apparatus, which may include beads with a high surface-to-volume ratio. As the serum incubates in the apparatus, DKK1 or other Wnt antagonists bind to the DKAB or other agent that specifically binds to DKK1 or to other Wnt antagonists. In the case of beads engineered to capture DKK1, the beads are incubated in direct contact with the serum or other ABM for a sufficient duration so as to effectively capture DKK1 (at least 15 minutes, and up to overnight), followed by separation or removal of the beads from the ABM via size exclusion (e.g. with a filter), or by a magnet in the case of magnetic or paramagnetic beads, or by other means recognized by those skilled in the art. If the agent that specifically binds DKK1 or other Wnt antagonist(s) is immobilized to surfaces within the apparatus that are not beads, the serum would be collected by means obvious to those skilled in the art, such as through a port, or by uptake in a syringe, or by expulsion by other means. The resulting DKK1-depleted serum can be applied to the scalp topically, or via subcutaneous injection into the scalp, or by other delivery methods. The product can also be combined with micronized adipose tissue (MAT) and injected with a dual syringe approach. The addition of the MAT provides the additional mesenchymal stem cells to stimulate further regeneration of the hair follicle. The DKK1-depleted serum (with or without adipose tissue) is expected to promote hair growth or thickness or to reduce hair loss, leading to improved esthetics. Such effects may be mediated by direct effects of serum-derived growth factors, which are enriched by virtue of obligatory platelet activation step of serum preparation (FIG. 1), and which may become more efficacious for hair restoration when serum-derived DKK1 and/or other Wnt antagonists present in serum are depleted via the apparatus. It follows that multiple binding factors could be immobilized on beads or elsewhere within the apparatus to capture and deplete multiple Wnt antagonists, thereby leading to further improvement in the hair-restoring effects of PRP.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

1. A composition comprising:
a promoter of Wnt signaling; and
an autologous body material, an allogeneic body material, a xenogeneic body material or a functional equivalent thereof.

2. The composition of claim 1, wherein the promoter of Wnt signaling comprises an anti-Dickkopf-related protein 1 (DKK1) antibody (DKAB) or other inhibitor of Dickkopf-related protein 1 (DKK1); scFv-DKK1c or other Wnt receptor heterodimerizing agent; a sclerostin antibody or other inhibitor of sclerostin; Wnt3a, liposomal Wnt3a or other recombinant Wnts that promote Wnt signaling; R-Spondin; lithium; WAY316606; LY2090314; promoters of LRP5, LRP6 or frizzled receptor signaling; an inhibitor of Kremen function; an inhibitor of Wnt inhibitory factor-1 (WIF-1); an inhibitor of Wise/SOSTDC; or an inhibitor of secreted frizzled-related proteins (sFRPs).

3. The composition of claim 1, wherein the autologous body material comprises platelet-rich plasma (PRP), platelet-rich fibrin (PRF), bone autograft, surgical bone, surgical blood, peripheral blood, hematoma, reamer-irrigator aspirate, plasma, platelet-poor plasma (PPP), bone marrow aspirate (BMA), bone marrow cell aspirate concentrate, or adipose derived ABM or a combination thereof, wherein the PRP, blood, plasma, PPP, and BMA may be in unclotted or clotted forms.

4. The composition of claim 1, wherein the promoter of Wnt signaling pathway is combined with, mixed with, entrapped in, encapsulated in, delivered alongside or is within the autologous, allogeneic or xenogeneic body material.

5. The composition of claim 2, wherein the promoter of Wnt signaling pathway is in a form of lyophilized, crystallized, liquid, frozen, gel, powder, paste or emulsion, prior to being combined with, mixed with, entrapped in, encapsulated in, delivered alongside or within the autologous, allogeneic or xenogeneic body material.

6. The composition of claim 1, comprising an anti-DKK1 antibody and platelet-rich fibrin or platelet-rich plasma.

7. The composition of claim 1, comprising an anti-DKK1 antibody and autologous bone graft or bone marrow, or bone marrow aspirate, or bone marrow aspirate concentrate, or reamer irrigated aspirate or any combination thereof.

8. The composition of claim 1, comprising an anti-DKK1 antibody and autologous plasma.

9. The composition of claim 1, comprising an anti-DKK1 antibody and autologous fibrin gel combined with a bone graft material.

10. A method of treating, or reducing the severity of, or reducing the likelihood of developing, or accelerating the healing of a defect or deficiency in a mammal by administering a promoter of Wnt signaling directly to locally expressed autologous body materials.

11. A method for treating, or reducing the severity of, or reducing the likelihood of developing, or accelerating the healing of a defect or deficiency in a mammal using the composition of claim 1, comprising:
obtaining a volume of an autologous body material from a mammal;
preparing the autologous body material;
adding a promoter of Wnt signaling to the prepared autologous body material to form a combined material; and
administering the combined material to the site of the defect or deficiency.

12. The method of claim 11, wherein a single-use kit is used in adding the promoter of Wnt signaling to the prepared autologous body material and further optionally in storing the combined material.

13. The method of claim 12, wherein the single-use kit comprises a sterile, closed system.

14. A device comprising the composition of claim 1.

15. The device of claim 14, further comprising an effective amount of an agent to induce platelet activation, fibrin formation, fibrin crosslinking or fibrin clot formation, which comprises one or more coagulant factors comprising calcium chloride, thrombin, prothrombin complex concentrate, chitosan, tissue factors, glass surface and silica.

16. The device of claim 14, adapted for a filler, a coating or an implant, wherein the implant comprises collagen sponge, a hydroxyapatite-containing sponge, a strip, a block, a plug, a bone graft, a dental implant, a craniofacial implant, an orthopedic implant, a prosthesis, a fusion cage, a screw, a plate, a pin, a button, a disc, a wire, or a rod.

17. A method of treating or reducing the severity of, or reducing the likelihood of developing, or accelerating the healing of a bone-related disease or condition, or cosmetically changing a bone structure in a subject, comprising:
administering the composition of claim 1 or implanting a device comprising said composition,
wherein the growth of new bone is promoted, or that damaged, injured or deficient bone is augmented, reconstituted, regenerated, fused, repaired, or healed.

18. The method of claim 17, wherein the disease or condition, or the bone structure change, comprises implant fixation, fracture repair, arthrodesis, extraction socket preservation, spinal fusion, bone healing, tendon or ligament reconstruction in bone, distraction osteogenesis, esthetic appearance and geometry, bone cavity defects, or traumatic bone loss.

19. The method of claim 18, wherein the composition is administered locally at a site of the disease or condition.

20. A method of treating or reducing the severity of, or reducing the likelihood of developing, or accelerating the healing of a wound related disease or condition, or esthetically changing the skin tissue in a subject, comprising:
administering the composition of claim 1 or implanting a device comprising said composition,
wherein the growth of new tissue is promoted, or a damaged, injured or deficient tissue is augmented, reconstituted, regenerated, repaired, or healed.

21. A method of treating or reducing the severity of, or reducing the likelihood of developing, or accelerating the reversal of a hair disease or disorder or condition, or esthetically changing the hair in a subject, comprising:
administering the composition of claim 1 or implanting a device comprising said composition,
wherein the growth or thickness of new hair is promoted, or a damaged, injured or deficient hair follicle is augmented, reconstituted, regenerated, repaired, or healed.

22. The method of claim 17, further comprising removing, reducing, or rendering inactive DKK1 or another inhibitor of Wnt signaling from the autologous body material prior to the administration or the implantation to the subject.

23. A composition or a device comprising a composition, wherein the composition comprises an autologous body material (ABM), or a functional equivalent thereof, wherein DKK1 or one or more other endogenous extracellular Wnt antagonists has been removed from or reduced in the ABM, wherein the Wnt antagonists comprise DKK1, sclerostin, secreted frizzled-related proteins (sFRPs), Wnt inhibitory factor 1 (WIF-1), Wise, or a combination thereof.

24. A method of preparing the composition of claim 23, wherein the DKK1 or other Wnt antagonist has been removed or reduced prior to, during, or after the preparation, the method comprising: obtaining an autologous body material (ABM) from a mammal, and isolating, concentrating, activating, polymerizing, cross-linking, and/or purifying the ABM.

25. A method of claim 24 wherein the DKK1 or other Wnt antagonist is removed or reduced using an apparatus that binds, filters, adheres to, eliminates, destroys, depletes or renders inactive DKK1 or other Wnt antagonists.

26. A method of claim 24 wherein platelets, growth factors, leucocytes or a combination thereof are substantially retained in the composition.

27. (canceled)

28. (canceled)
